(19) United States
(12) Patent Application Publication
(10) Pub. No.: US 2023/0381348 A1
(43) Pub. Date: Nov. 30, 2023

(54) BIFUNCTIONAL MACROCYCLIC CHELATE, CONJUGATE, METAL COMPLEX AND USE THEREOF

(71) Applicant: Jyams Pet Research & Development Limited, Jiangsu (CN)

(72) Inventors: Jianfeng Xu, Jiangsu (CN); Zheng Wang, Jiangsu (CN); Wenbin Fan, Jiangsu (CN); Fei Cai, Jiangsu (CN); Huanhuan Yin, Jiangsu (CN); Zhigang Luo, Jiangsu (CN)

(73) Assignee: Jyams Pet Research & Development Limited, Jiangsu (CN)

(21) Appl. No.: 18/362,729

(22) Filed: Jul. 31, 2023

Related U.S. Application Data
(63) Continuation-in-part of application No. PCT/CN2021/130732, filed on Nov. 15, 2021.

(30) Foreign Application Priority Data
Feb. 1, 2021 (CN) 202110139386.4
Jul. 30, 2021 (CN) 202110872439.3

(51) Int. Cl. A61K 51/04; A61K 51/08; A61K 51/10; A61K 51/06; A61P 35/00

(52) U.S. Cl. CPC A61K 51/0482; A61K 51/088; A61K 51/1021; A61K 51/065; A61P 35/00

(57) ABSTRACT

The present disclosure relates to the technical field of chelating agents, in particular to a bifunctional macrocyclic chelate, a conjugate, a metal complex, and use thereof. The bifunctional macrocyclic chelate is a compound of following Formula I or an isomer thereof,

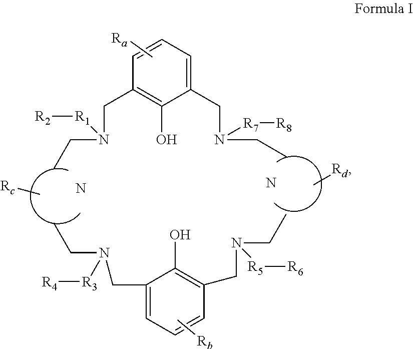

wherein R₁, R₃, R₅, and R₇ are each independently selected from alkyls or long-chain polymer groups, R₂, R₄, R₆, and R₈ are each independently selected from functional groups, Rₐ, R_{b}, R_{c}, and R_{d} are each independently selected from any one of H, substituted or unsubstituted alkyls, halogens, substituted or unsubstituted cyano groups, substituted or unsubstituted ester groups, substituted or unsubstituted alkoxys, substituted or unsubstituted amines, and substituted or unsubstituted aryls, ring

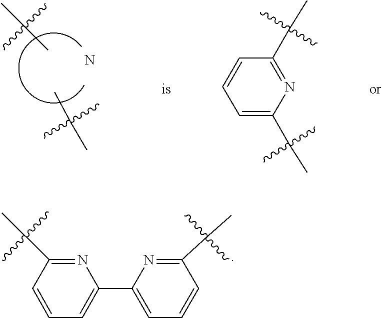

The bifunctional macrocyclic chelate can complex most nuclides, and expand use of the bifunctional macrocyclic chelate, and the metal complex chelating different nuclides can realize integration of diagnosis and treatment.

### CROSS-REFERENCE TO RELATED APPLICATIONS

The present disclosure is a continuation-in-part application of International Patent Application No. PCT/CN2021/130732, filed Nov. 15, 2021, entitled “Bifunctional Macrocyclic Chelate, Conjugate, Metal Complex and Use thereof,” which claims the benefit of CN 202110139386.4, filed Feb. 1, 2021, which also claims the benefit of CN 202110872439.3, filed Jul. 30, 2021, the contents of which are incorporated herein by reference in their entirety.

### TECHNICAL FIELD

The present disclosure relates to the technical field of chelating agents, and in particular to a bifunctional macrocyclic chelate, a conjugate, a metal complex, and use thereof.

### BACKGROUND ART

Radioactive metal nuclides usually need to be linked with target molecules through bifunctional chelating agents to form radioactive probes. Stability of the radioactive probes in vivo is an important criterion for judging its performance. As the radioactive metal nuclides have respective advantages and disadvantages, there are no ideal radioactive nuclides. It is necessary to consider a good number of aspects in combination with application purpose prior to selecting a metal nuclide. Common chelating agents in the prior art can only specifically chelate one or several radionuclides, and are unable to chelate radionuclides in broad spectrum. For example, DOTA can chelate diagnostic nuclides ⁶⁸Ga (t_{1/2}=68.1 min), ¹¹¹In, and therapeutic nuclide ¹⁷⁷Lu, whereas DOTA cannot chelate Al[¹⁸F] (t_{1/2}=109.8 min) and ⁸⁹Zr (t_{1/2}=78.4 h). Thus it has to be replaced by other chelating agents, for example, NOTA chelates Al[¹⁸F], and DFO chelates ⁸⁹Zr. Universal chelating agents are lacked in current research.

Secondly, as for FDA approved integrated scanning images for diagnosis and treatment, such as ⁶⁸Ga-DOTATATE & ¹⁷⁷Lu-DOTATATE, multiple administrations are required when monitoring the efficacy of ¹⁷⁷Lu-DOTATATE because of a relatively short half-life period of ⁶⁸Ga. Compared with ⁶⁸Ga, ⁸⁹Zr has a relatively long half-life period, and can achieve long-term evaluation and monitoring after a single administration. Meanwhile, dose analysis of the ⁸⁹Zr scanning image, could achieve the calculation of absorbed doses by tumors and normal organs, thereby fulfilling the possibility of accurately guiding the dosage of therapeutic medicine. Nonetheless, a relatively high temperature is required when DOTA chelates ⁸⁹Zr, which may deactivate the target molecule. On the other hand, a bifunctional chelating agent commonly used for ⁸⁹Zr is DFO. Thus, if it is attempted to use ⁸⁹Zr to evaluate the effect of a therapeutic medicine for treating ¹⁷⁷Lu and the like, the precursor to be labeled (namely, chelating agent-target molecule) needs to be replaced, then the purpose of integration of diagnosis and treatment cannot be precisely achieved.

### SUMMARY

The present disclosure provides a bifunctional macrocyclic chelate, which is a compound of the following Formula I or an isomer thereof,

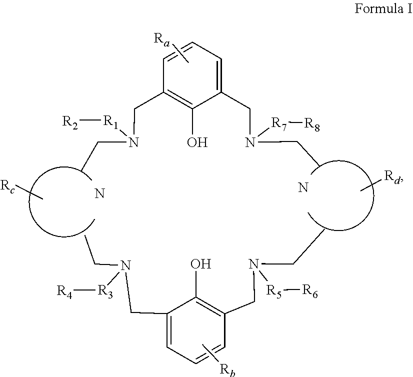

wherein R₁, R₃, R₅, and R₇ are each independently selected from substituted or unsubstituted alkyls or long-chain polymer groups,

R₂, R₄, R₆, and R₈ are each independently selected from functional groups capable of reacting with the target molecule and capable of coordinating with a metal ion,

Rₐ, R_{b}, R_{c}, and R_{d} are each independently selected from any one of H, substituted or unsubstituted alkyls, halogens, substituted or unsubstituted cyano groups, substituted or unsubstituted ester groups, substituted or unsubstituted alkoxys, substituted or unsubstituted amino groups, and substituted or unsubstituted aryls, and

ring

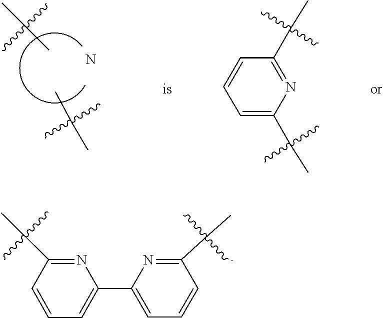

The present disclosure provides a conjugate, wherein the conjugate is a compound formed by coupling of a target molecule with at least one group of R₂, R₄, R₆, and R₈ in the bifunctional macrocyclic chelate of any of the previous embodiments.

The present disclosure provides a metal complex, wherein the metal complex is a complex formed by chelation of the bifunctional macrocyclic chelate of any one of the preceding embodiments or the conjugate of any one of the preceding embodiments with a metal ion.

The present disclosure provides use of the bifunctional macrocyclic chelate of any one of the preceding embodiments or the conjugate of any one of the preceding embodiments or the metal complex of the preceding embodiment in the preparation of a medicine for diagnosis and/or treatment of tumors.

The present disclosure provides use of the bifunctional macrocyclic chelate of any one of the preceding embodiments or the conjugate of any one of the preceding embodiments or the metal complex of the preceding embodiment in the preparation of a radioactive drug.

Optionally, the radioactive drug is a diagnostic agent for molecular imaging.

Optionally, the radioactive drug is a medicine that can treat tumors.

The present disclosure further provides a method of treating tumors, including administering a therapeutically effective amount of the metal complex of any of the preceding embodiments to a subject in need thereof.

The present disclosure further provides a method of imaging, including administering a diagnostically effective amount of the metal complex of any of the preceding embodiments to a subject in need thereof, exposing the subject to a scanning device, and obtaining a scanning image of the subject.

The present disclosure has the following beneficial effects: the bifunctional macrocyclic chelate provided in the embodiments of the present disclosure has four linkers, which can be coupled with one or more target molecules, to achieve more precise targeted diagnosis or treatment. Meanwhile, the bifunctional macrocyclic chelate has relatively high selectivity and coordination capacity with divalent, trivalent, or tetravalent metal ions, and can form stable metal complexes with them. The metal complexes formed have good in vivo and in vitro stability, can specifically target tumors, and are mainly excreted through kidneys. The bifunctional macrocyclic chelate can complex most of the diagnostic or therapeutic metal ions, to achieve integration of radioactive diagnosis and treatment. Furthermore, diagnostic radiopharmaceuticals with a relatively long half-life period can also provide the possibilities of clinical efficacy monitoring and medication guidance for therapeutic medicines.

### BRIEF DESCRIPTION OF DRAWINGS

In order to more clearly illustrate technical solutions of examples of the present disclosure, drawings which need to be used in the examples will be introduced below briefly, and it should be understood that the drawings below merely show some examples of the present disclosure, and therefore should not be considered as limitation to the scope, and a person ordinarily skilled in the art still could obtain other relevant drawings according to these drawings, without using any creative efforts.

FIG. 1 is a mouse PET scanning image in Experimental Example 1 of the present disclosure;

FIG. 2 is a mouse PET scanning image in Experimental Example 2 of the present disclosure;

FIG. 3 is a mouse PET scanning image in Experimental Example 3 of the present disclosure;

FIG. 4 shows a drug-time curve obtained from pharmacokinetic experiment in Experimental Example 4 of the present disclosure;

FIG. 5 shows results of binding rate, internalization rate, and efflux rate of various metal complexes measured in Experiment A of Experimental Example 5 of the present disclosure;

FIG. 6 shows results of binding rate, internalization rate, and efflux rate of various metal complexes measured in Experiment B of Experimental Example 5 of the present disclosure;

FIG. 7 is a SPECT scanning image of LNCap tumor-bearing mouse animal models in Experimental Example 6 of the present disclosure;

FIG. 8 is a curve chart of tumor size changes of the LNCap tumor-bearing mouse animal models in Experimental Example 6 of the present disclosure;

FIG. 9 is a curve chart of body weight changes of the LNCap tumor-bearing mouse animal models in Experimental Example 6 of the present disclosure; and

FIG. 10 is a mouse PET scanning image in Experimental Example 7 of the present disclosure.

### DETAILED DESCRIPTION OF EMBODIMENTS

In order to make the objectives, technical solutions, and advantages of the embodiments of the present disclosure clearer, the technical solutions in the embodiments and examples of the present disclosure will be described below clearly and completely. If no specific conditions are specified in the embodiments and examples, they are carried out under normal conditions or conditions recommended by the manufacturers. If manufacturers of reagents or apparatuses used are not specified, they are conventional products commercially available. In the above, the antibody KN035 was provided by Alphamab Oncology Co. Ltd, Suzhou.

The present disclosure provides a bifunctional macrocyclic chelate, a conjugate, a metal complex, and use thereof. The bifunctional macrocyclic chelate provided in the embodiments of the present disclosure can chelate most nuclides in broad spectrum, and extend use of the bifunctional macrocyclic chelate, and the metal complex chelating different nuclides can realize integration of diagnosis and treatment.

The present disclosure provides a bifunctional macrocyclic chelate, which is a compound of the following Formula I or an isomer thereof,

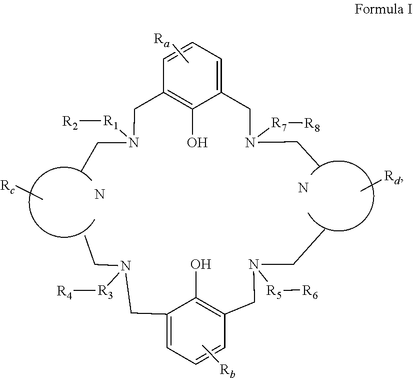

wherein

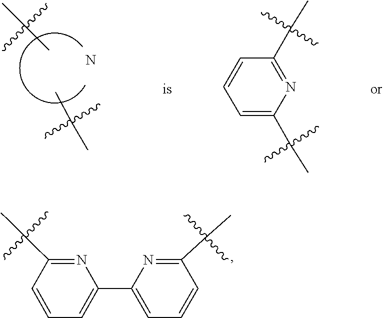

R₁, R₃, R₅, and R₇ are each independently selected from substituted or unsubstituted alkyls or long-chain polymer groups, and the main purpose of R₁, R₃, R₅, and R₇ is to increase the chain length, so as to facilitate the subsequent interaction of the target molecule with the bifunctional macrocyclic chelate.

In some embodiments, R₁, R₃, R₅, and R₇ are each independently selected from straight-chain alkyls, and may be unsubstituted straight-chain alkyls, such as C1-C20 unsubstituted straight-chain alkyls, and is (CH₂)ₙ in some embodiments, wherein n is selected from 1-8, such as methylene.

It can be certainly appreciated that other unsubstituted straight-chain alkyls are also within the scope of protection of the present disclosure, such as C16 straight-chain alkyl, and C18 straight-chain alkyl. Meanwhile, it can also be a non-straight-chain alkyl, such as a branch chain formed, which does not affect subsequent coupling with target molecule or complexation with the metal ion, and the branched alkyl can also be used. Further, the alkyls may be substituted alkyls, for example, they may be alkyls substituted with a substituent such as halogen, cyano group, ester group, and ether group.

The long-chain polymer groups refer to groups formed by polymers, and may be optionally straight long-chain polymer groups, can be any one of straight long-chain polymer groups containing amide groups and/or PEG groups (namely, polyethylene glycol groups), that is, the long-chain polymer groups may be other straight long-chain polymer groups containing amide groups and without PEG chain, straight long-chain polymer groups containing PEG chain and without amide group, or straight long-chain polymer groups simultaneously containing amide groups and PEG chain. Moreover, the number of amide groups may be one, two, three or more. Meanwhile, the long-chain polymer groups may also be other long-chain polymers capable of extending the chain length thereof, which do not affect or do enhance the subsequent coupling of the bifunctional macrocyclic chelate with the target molecule and complexing action with metal ion.

R₂, R₄, R₆, and R₈ are each independently selected from functional groups capable of reacting with the target molecule and capable of coordinating with the metal ion, wherein the functional groups may be any one of amino groups, acid radical groups capable of forming organic acids, derivative groups of the amino groups, and derivative groups of the acid radical groups, and the derivative groups refer to derivative groups formed by further substitution and derivation of the amino or acid radical groups. In some embodiments, R₂, R₄, R₆, and R₈ are each independently selected from any one of amino groups, carboxyls, sulfonic acid groups, phosphoric acid groups, thiol groups, amide groups, sulfonamide groups, isothiocyanate groups, tetrafluorophenol groups, and succinimide groups.

It should be certainly understood that the functional groups may also be selected according to the target molecule and the metal ion, and other groups may also be used, as long as they have the function of reacting with the target molecule and coordinating with the metal ion.

Further, each of Rₐ and R_{b} represents one or more substituents at any position(s) on the corresponding phenol ring, and the substituent is not limited to one, i.e., may be multi-substitutions, for example, any one of para-position and meta-positions of the OH can be substituted, and it is also possible that two meta-positions of the OH are both substituted, the para-position and one meta-position are simultaneously substituted, or all of the para-position and/or two meta-positions of the OH are substituted, and in cases of multi-substitutions, each substituent may be identical or different.

Each of R_{c} and R_{d} represents one or more substituents at any position of the ring

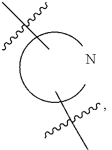

i.e., pyridine ring or bipyridine ring, and the number of substituents is not limited to one, that is, may be multi-substitutions.

Further, Rₐ, R_{b}, R_{c}, and R_{d} are each independently selected from any one of H, substituted or unsubstituted alkyls, halogens, substituted or unsubstituted cyano groups, substituted or unsubstituted ester groups, substituted or unsubstituted alkoxys, substituted or unsubstituted amino groups, and substituted or unsubstituted aryls. Rₐ, R_{b}, R_{c}, and R_{d} are each independently selected from H, halogens, C1-C20 substituted or unsubstituted alkyls, substituted or unsubstituted C1-C20 cyano groups, substituted or unsubstituted C1-C20 alkoxys, substituted or unsubstituted C2-C20 ester groups, substituted or unsubstituted C2-C20 amino groups, substituted or unsubstituted C3-C20 heteroaryls and C4-C20 non-heteroaryls, optionally, Rₐ, R_{b}, R_{c}, and R_{d} are each independently selected from any one of H, methyl, ethyl, n-propyl, iso-propyl, n-butyl, iso-butyl, t-butyl, halogens, methoxy, ethoxy, substituted or unsubstituted phenyls, substituted or unsubstituted benzyls, tertiary amino groups, cyano groups, substituted or unsubstituted thienyls, and substituted or unsubstituted benzofuranyls.

In some embodiments, the bifunctional macrocyclic chelate is any one selected from compounds of following structural formulas:

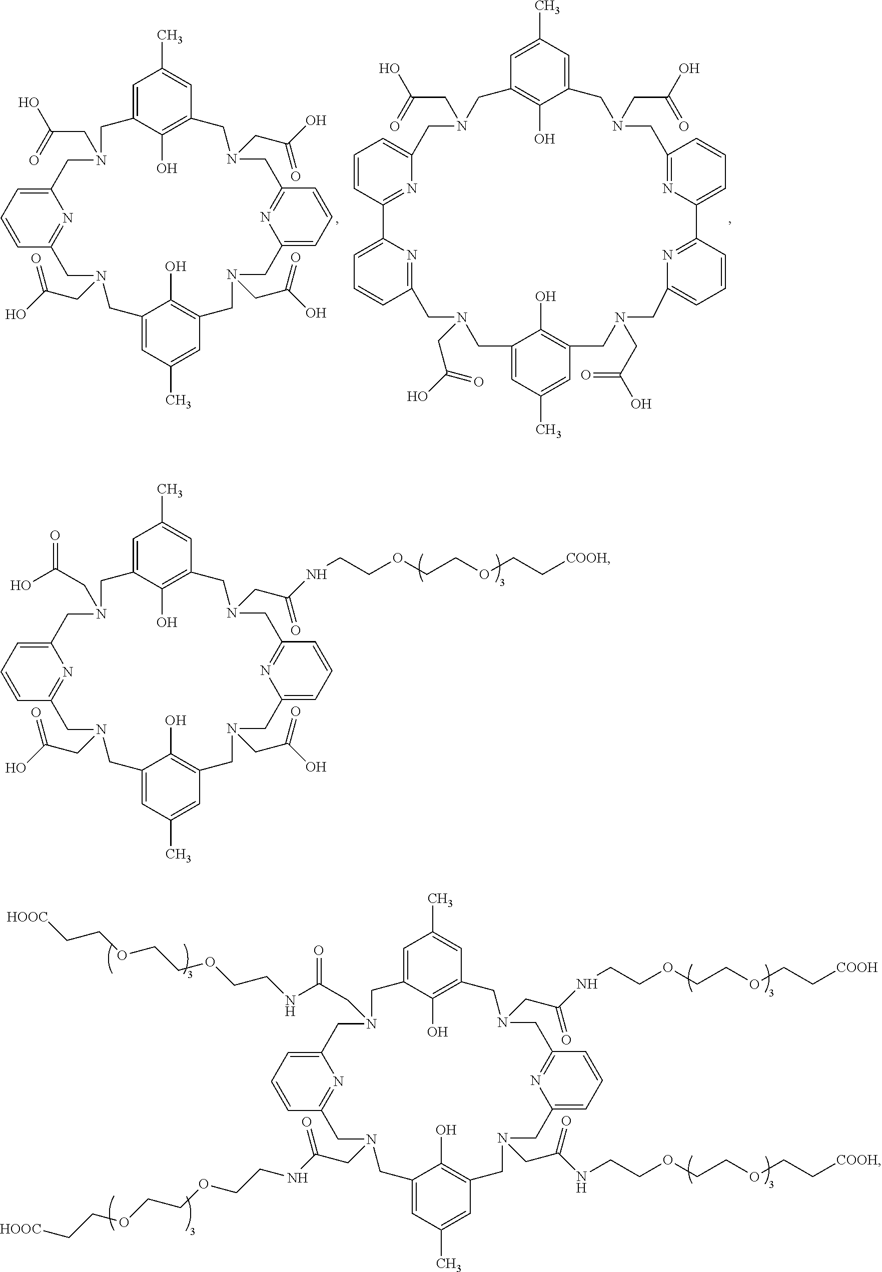

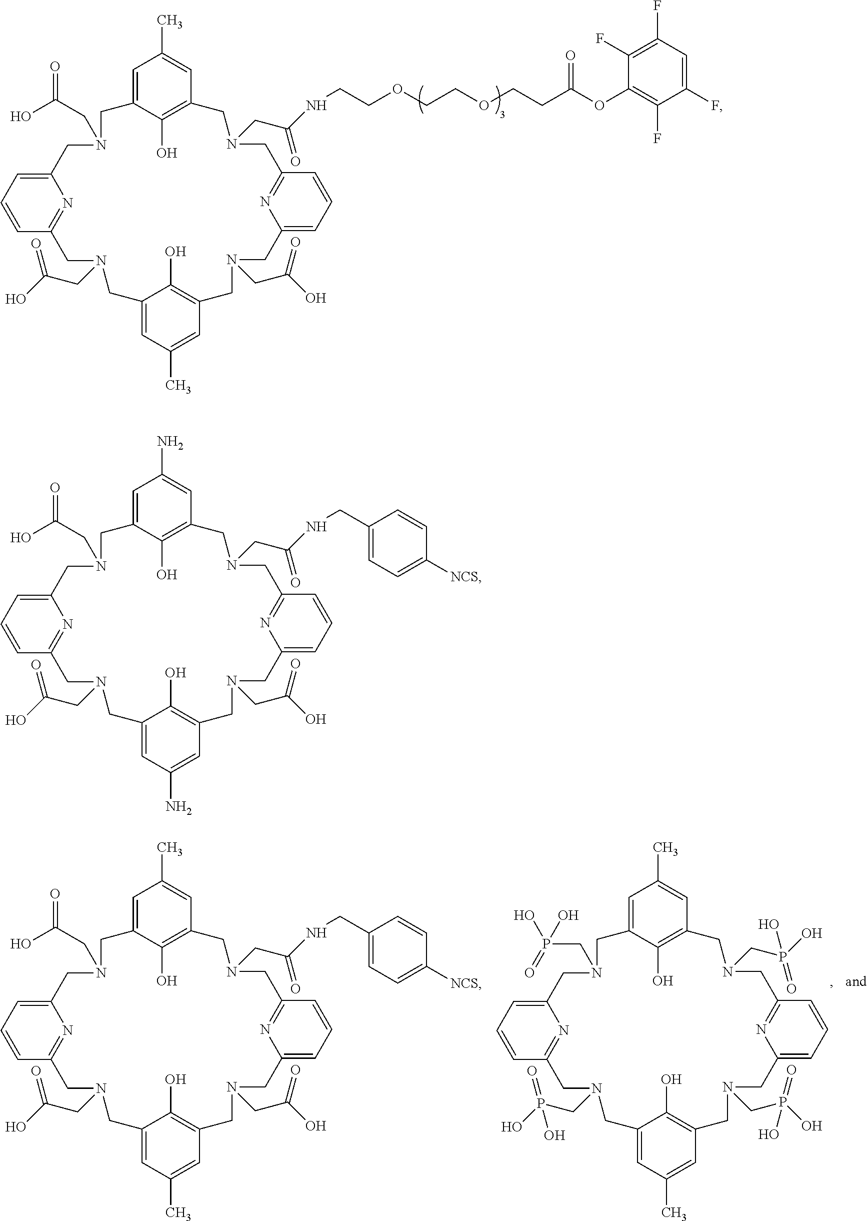

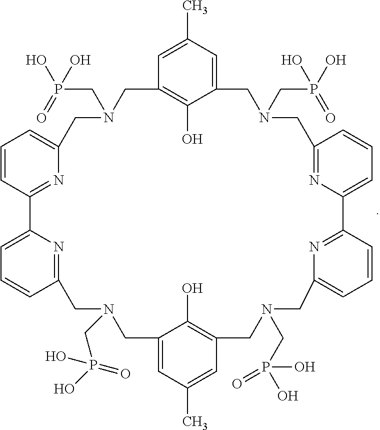 [SMILES: Cc1cc2c(O)c(c1)CN(CP(=O)(O)O)Cc1cccc(n1)-c1cccc(n1)CN(CP(=O)(O)O)Cc1cc(C)cc(c1O)CN(CP(=O)(O)O)Cc1cccc(n1)-c1cccc(n1)CN(CP(=O)(O)O)C2]

An embodiment of the present disclosure further provides a conjugate, wherein the conjugate is a compound formed by coupling a target molecule with at least one group of R₂, R₄, R₆, and R₈ in the above bifunctional macrocyclic chelate. In some embodiments, the target molecule can be coupled with any group of R₂, R₄, R₆, and R₈, for example, coupled with R₆, R₂, R₄, or R₅ respectively. The target molecule also can be coupled with any two groups of R₂, R₄, R₆, and R₈, such as R₂ and R₆, R₂ and R₄, R₂ and R₈, R₄ and R₆, R₄ and R₈, or R₆ and R₈. Alternatively, the target molecule also can be coupled with any three groups of R₂, R₄, R₆, and R₈, for example simultaneously with R₂, R₄, and R₆, simultaneously with R₂, R₄, and R₈, simultaneously with R₂, R₆, and R₈, and even simultaneously with R₂, R₄, R₆, and R₈.

The essence of coupling is that corresponding group has corresponding reaction with the target molecule, and then the target molecule is bonded to the bifunctional macrocyclic chelate. In some embodiments, the coupling process comprises: reacting the bifunctional macrocyclic chelate with a condensing agent and the target molecule under a condition of 0-100° C., wherein pH of a reaction system of the above reaction is 2-11, and the condensing agent is at least one of HOAt, HOBt, HATU, HBTU, DMAP, PyBOP, EDC, DCC, DIC, and NHS; and the solvent is at least one of DMF, THF, DMSO, and DCM. Utilization of the above condition facilitates the coupling.

Further, the target molecule may be any one selected from antibodies, proteins, peptides, carbohydrates, nucleotides, oligonucleotides, oligosaccharides, vitamins, liposomes, small-molecule drugs or fragments or derivatives thereof; and optionally, the target molecule is any one of antibodies (e.g., antibodies with target of PD-L1, PD-1, FAP, PSMA, VEGF, EGFR, CD-X, HER2, HER3, or CEA), peptides (which may be, for example, RGD peptides or peptides targeting PSMA, FAP, or SSTR), and vitamins (which may be, for example, folic acid).

An embodiment of the present disclosure further provides a metal complex, wherein the metal complex is a complex formed by chelation of the above bifunctional macrocyclic chelate or the conjugate of any one of the preceding embodiments with a metal ion. In the above, the metal ion is a radioactive ion or a non-radioactive ion; for example, the radioactive ion is selected from any one of Al[¹⁸F], ⁵¹Mn, ^{52m}Mn, ^{52g}Mn, ⁶⁴Cu, ⁶⁷Cu, ⁶⁷Ga ⁶⁸Ga, ⁸⁹Zr, ⁸⁶Y, ⁹⁰Y, ^{99m}Tc, ¹¹¹In, ¹⁵³Sm, ¹⁶⁶Ho, ¹⁷⁷Lu, ¹⁸⁶Re, ¹⁸⁸Re, ²¹¹At, ²¹²Bi, ²¹²Pb, ²¹³Bi, ²²³Ra, ²²⁵Ac, and ²²⁷Th.

Moreover, the chelation process comprises: mixing a solution containing the conjugate with a solution containing the metal ion, adjusting pH of the solution to 3-11, and performing reaction under a condition of 0-110° C. Optionally, pH is 4-9. Utilization of the above condition facilitates the formation of the complex.

An embodiment of the present disclosure provides use of the above bifunctional macrocyclic chelate or the above conjugate or the above metal complex in the preparation of a medicine for diagnosis and/or treatment of tumors.

An embodiment of the present disclosure further provides use of the above bifunctional macrocyclic chelate or the above conjugate or the above metal complex in the preparation of a radioactive drug, wherein the radioactive drug is a diagnostic agent of molecular imaging and a medicine that can treat tumors.

An embodiment of the present disclosure further provides a method of treating tumors, including administering a therapeutically effective amount of the above metal complex to a subject in need thereof.

An embodiment of the present disclosure further provides a method of imaging, including administering a diagnostically effective amount of the above metal complex to a subject in need thereof, exposing the subject to a scanning device, and obtaining a scanning image of the subject.

The features and performances of the present disclosure are further described in detail below in combination with examples.

### Example 1

The present example provides a bifunctional macrocyclic chelate (denoted as Dar), which has a structural formula as follows:

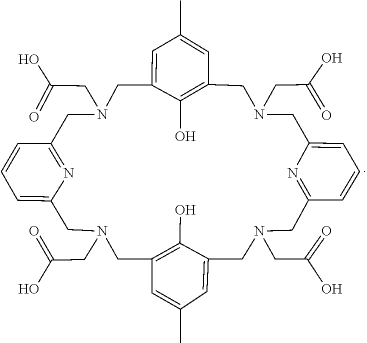 [SMILES: Cc1cc2c(O)c(c1)CN(CC(=O)O)Cc1cccc(n1)CN(CC(=O)O)Cc1cc(C)cc(c1O)CN(CC(=O)O)Cc1cccc(n1)CN(CC(=O)O)C2]

The present example further provides a preparation method for the above bifunctional macrocyclic chelate, wherein the synthesis is carried out according to a synthetic route as follows:

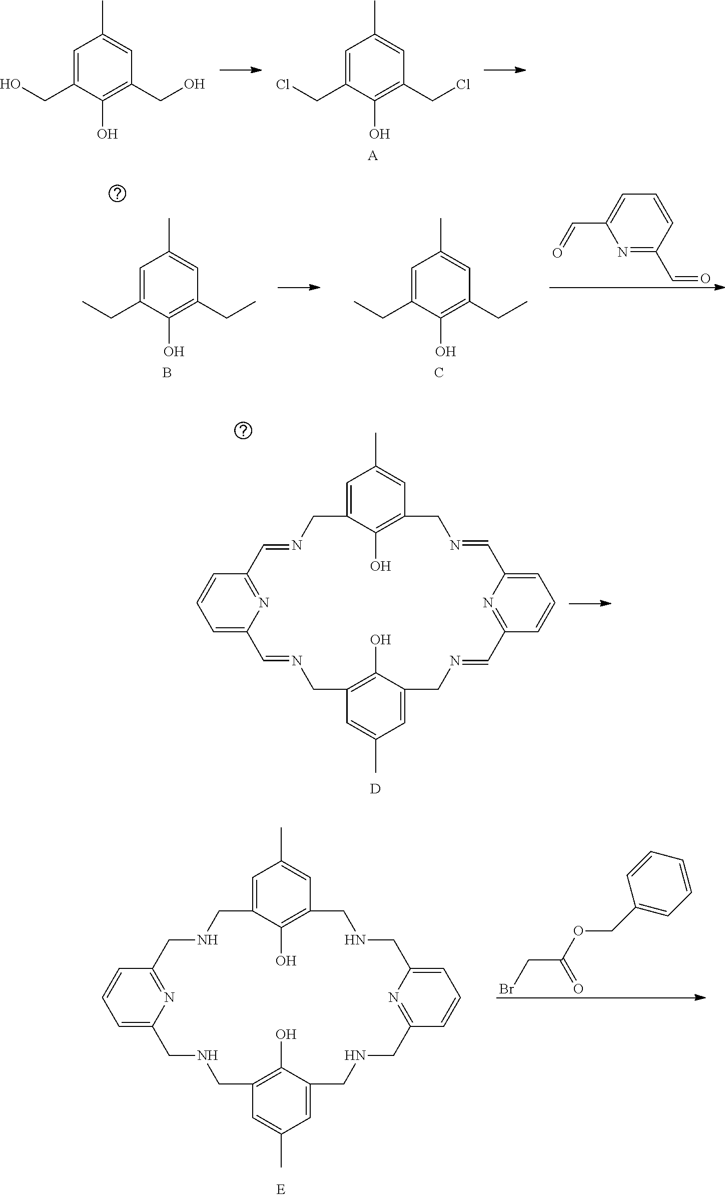

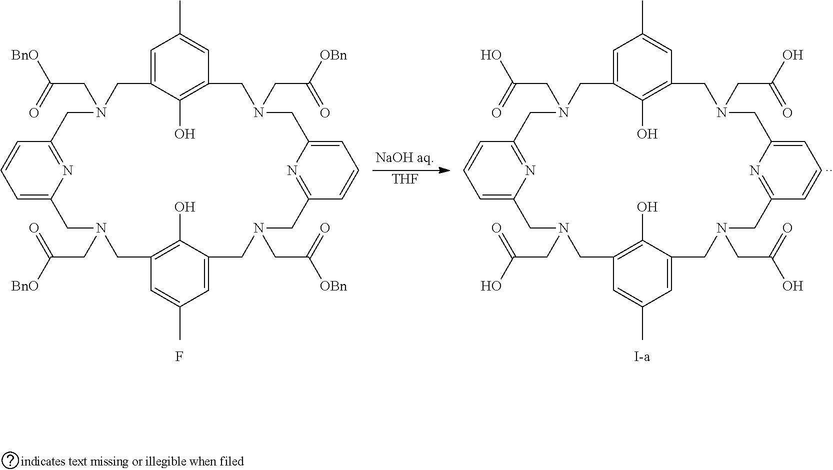

The experimental operations are as follows:

Step 1:

2,6-Bis(hydroxymethyl)-4-methylphenol (16.8 g, 0.1 mol) was dissolved in dichloromethane (20 mL). Concentrated hydrochloric acid (100 mL) was added, and the mixture was stirred at room temperature for 24 h. The reaction mixture was concentrated to dryness to afford Compound A.

Step 2:

A methanol (380 mL) solution of urotropine (18.9 g, 0.14 mol) was swiftly added at room temperature to the freshly dissolved Compound A (0.061 mol) vigorously stirred. After several seconds, a fine colorless microcrystalline solid was separated. The mixture was allowed to stand at room temperature for 15 min, and the solid was collected, washed with cold methanol, and dried in vacuo at room temperature. 20.67 g of crude Compound B was obtained.

A suspension of Compound B (10.2 g) in a mixture of ethanol (20 mL) and concentrated hydrochloric acid aqueous solution (14 mL, 32%) was heated to the boiling point, and stirred continuously for 2 h. Additional concentrated hydrochloric acid aqueous solution (10 mL, 32%) was added, and the mixture was stirred continuously for 2 h. The mixture was dried in vacuo to afford a mixture of ammonium chloride and Compound C. Ethanol (83 mL) was added and the mixture was stirred overnight. After filtration, n-hexane (83 mL) was added to the filtrate, and the mixture was stirred overnight. A pale yellow precipitate was collected and dried in vacuo to afford 1.4 g of Compound C.

Step 3:

To a methanol (75 mL) solution of Compound C (482.2 mg, 1.9 mmol) was added DIPEA (620 μL, 3.8 mmol), and then the mixture was heated to 55° C. A methanol (63 mL) solution of 2,6-diformylpyridine (253.45 mg, 1.88 mmol) was added dropwise to the above mixture at 55° C., and the resultant mixture was stirred for 2 h. Then the solution was stirred overnight at room temperature. A white precipitate was obtained by filtration and dried in vacuo to afford 374.2 mg of Compound D.

Step 4:

A suspension of Compound D (182.6 mg) in methanol (30 mL) was heated to 45° C. NaBH₄ (421.7 mg) was added in batches, and the mixture was stirred at 45° C. for 3 h. The solvent was evaporated under reduced pressure, and the product was extracted with dichloromethane (CH₂Cl₂/H₂O, 2×20 mL/20 mL). The organic phase was dried with anhydrous Na₂SO₄ and then filtered. After the solvent of dichloromethane was removed in vacuo, about 2 mL of methanol solution of 36% HCl was added. The resultant mixture was left to stand at 4° C. overnight, and a white precipitate was formed. The precipitate was filtered and dried to afford the pure hydrochloride of Compound E.

Step 5:

Compound E (716.4 mg, 0.907 mmol, 1 eq), benzyl bromoacetate (1430 μL, 9.0 mmol, 10 eq), DIPEA (1865 μL, 11.284 mmol, 12 eq), and acetonitrile (ACN, 9.5 mL) were mixed, and stirred at room temperature (around 25° C.) for 2 h. The mixture was subjected to rotary evaporation to dryness, and then column chromatography followed by rotary evaporation to dryness afforded a pale grey foamy solid Compound F.

LC-MS: 566.4 [M/2+H]⁺.

Step 6:

Compound F (1 eq), NaOH solution (5.3 mL, 10 M, 15 eq), deionized water (22 mL), and THF (27 mL) were mixed, and stirred in oil bath at 60° C. overnight, a white solid was precipitated, and was subjected to suction filtration and drying to afford a sodium salt of Compound I-a, and pH was adjusted to 6-7 with hydrochloric acid, to afford Compound I-a, i.e., bifunctional macrocyclic chelate Dar.

Structural characterization data of the bifunctional macrocyclic chelate are as follows:

¹HNMR (400 MHz, D₂O): δ 7.48 (t, J=7.8 Hz, 2H), 7.02 (d, J=7.8 Hz, 4H), 6.86 (s, 4H), 3.62 (s, 8H), 3.55 (s, 8H), 3.12 (s, 8H), 2.11 (s, 6H).

LC-MS: 386.3[M/2+H]⁺, 771.3[M+H]⁺, 793.4[M+Na]⁺.

### Example 2

The present example provides a bifunctional macrocyclic chelate, which has a structural formula as follows:

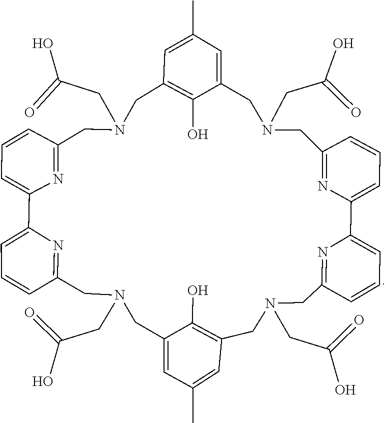 [SMILES: Cc1cc2c(O)c(c1)CN(CC(=O)O)Cc1cccc(n1)-c1cccc(n1)CN(CC(=O)O)Cc1cc(C)cc(c1O)CN(CC(=O)O)Cc1cccc(n1)-c1cccc(n1)CN(CC(=O)O)C2]

The present example further provides a preparation method for the above bifunctional macrocyclic chelate, wherein synthesis is carried out according to a synthetic route as follows:

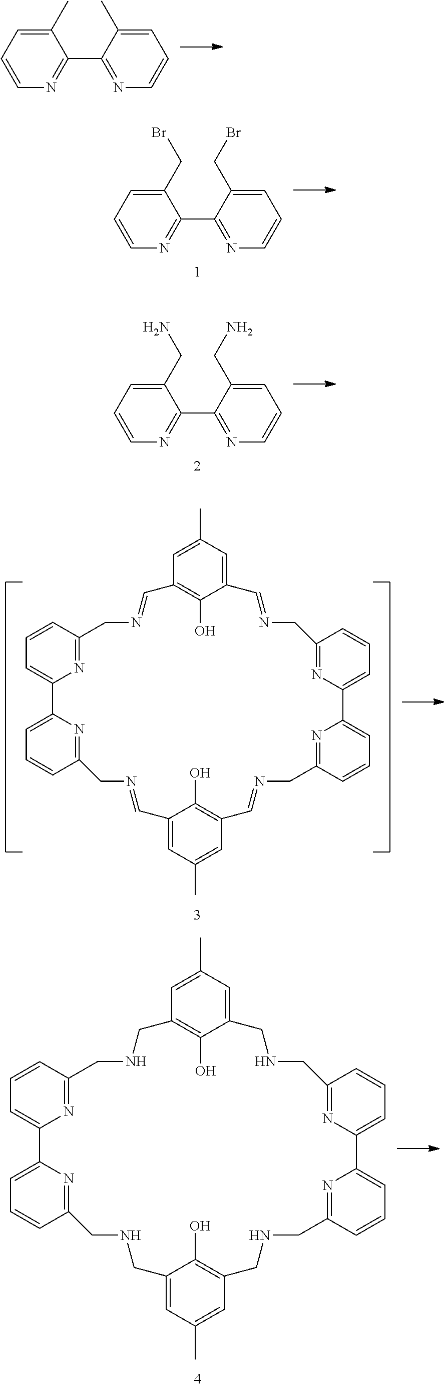

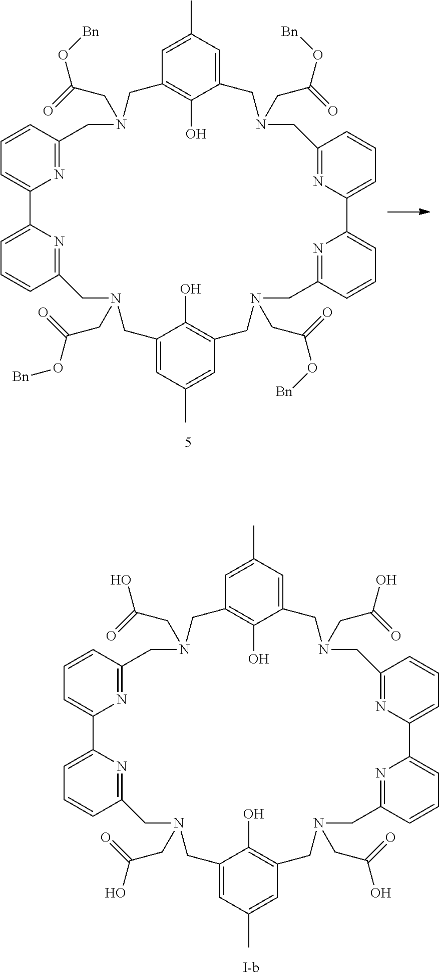 [SMILES: Cc1cc2c(O)c(c1)CN(CC(=O)OCc1ccccc1)Cc1cccc(n1)-c1cccc(n1)CN(CC(=O)OCc1ccccc1)Cc1cc(C)cc(c1O)CN(CC(=O)OCc1ccccc1)Cc1cccc(n1)-c1cccc(n1)CN(CC(=O)OCc1ccccc1)C2 ; Cc1cc2c(O)c(c1)CN(CC(=O)O)Cc1cccc(n1)-c1cccc(n1)CN(CC(=O)O)Cc1cc(C)cc(c1O)CN(CC(=O)O)Cc1cccc(n1)-c1cccc(n1)CN(CC(=O)O)C2]

The experimental operations are as follows:

Step 1:

6,6′-Dimethyl-2,2′-bipyridine (22.1 g), NBS (44.7 g), and benzoyl peroxide (250 mg) were added to 1200 mL of carbon tetrachloride solution, the mixture was refluxed for 6 h, and subjected to hot filtration, the filtrate was cooled to 0-10° C. and maintained, followed by suction filtration. To the solid obtained was added 100 mL of methanol for pulping, followed by suction filtration, and the solid was dried to afford 6.91 g of Compound 1.

LC-MS: 340.9[M+H]⁺.

Step 2:

Compound 1 (4.25 g) was dissolved in 160 mL of chloroform, the mixture was heated and refluxed for dissolution, and 3.8 g of hexamethylenetetramine was dissolved in 100 mL of chloroform. The mixture was added dropwise under reflux to the reaction system. After the dropwise addition was completed, the mixture was refluxed for 4 h. After being cooled to room temperature, the mixture was subjected to suction filtration, the filter cake was dried in vacuo, the obtained solid was dissolved in a solution of water (30 mL)/ethanol (150 mL)/hydrochloric acid (35 mL), the mixture was heated and refluxed until fully dissolved, the resultant mixture was cooled to room temperature for precipitation, suction filtration, and drying to afford 4.40 g of Compound 2 (hydrochloride). Sodium hydroxide (48.37 g) was dissolved in 250 mL of purified water, 20.10 g of Compound 2 (hydrochloride) was added and the mixture was stirred to dissolve, aqueous phase was extracted with 3×400 mL of dichloromethane, organic phases were combined and washed with saturated brine, dried with anhydrous sodium sulfate, and subjected to rotary evaporation to dry to afford 11.97 g of Compound 2. The obtained Compound 2 was dissolved by adding 800 mL of methanol for subsequent use.

LC-MS: 215.1 [M+H]⁺.

Step 3:

To lanthanum acetate (21.34 g) and 2-hydroxy-5-methylisophthalaldehyde (10.06 g) was added 1.8 L of methanol, the mixture was heated and refluxed until solids were completely dissolved, and under reflux, the methanol solution of Compound 2 was slowly added dropwise to the reaction system and refluxed overnight. After being cooled to room temperature, reaction solution was subjected to suction filtration, filtrate was subjected to rotary evaporation until about half of the volume was left, the resultant mixture was heated to 40° C., sodium cyanoborohydride (22.01 g) and sodium borohydride (35.28 g) were added in batches, after the addition was completed, the mixture was heated and refluxed overnight, reaction solution was subjected to rotary evaporation to dry, hydrochloric acid (140 mL, 6M) was added and the mixture was stirred well, DTPA solution (96.30 g of DTPA was dissolved in 1000 mL of purified water) was added, and the mixture was stirred overnight. A sodium hydroxide solution was added to adjust pH to 8-9, the aqueous phase was extracted with 3×500 mL of dichloromethane, and organic phases were collected and combined, washed with saturated brine, dried with anhydrous sodium sulfate, and subjected to suction filtration and rotary evaporation to dry to afford 5.89 g of Compound 4.

LC-MS: 347.2[M/2+H]⁺, 693.4 [M+H]⁺.

Step 4:

To Compound 4 (3.46 g) were added 50 mL of dichloromethane and the mixture was stirred, triethylamine (8.15 g) was added, and benzyl bromoacetate (5.74 g) was added dropwise at room temperature to the reaction system, the mixture was allowed to react at room temperature overnight, tracked by TLC (MeOH:DCM=1:10). When raw materials reacted completely, 50 mL of water was added for liquid separation, the aqueous phase was extracted with 2×50 mL of dichloromethane, organic phases were combined and washed with water, washed with saturated brine, dried with anhydrous sodium sulfate, subjected to suction filtration and rotary evaporation to dry, and the obtained oily substance was separated and purified twice by column chromatography (MeOH:DCM=100:1-100:3), and the less polar products displayed by TLC were collected and subjected to rotary evaporation to dry to afford 1.06 g of Compound 5.

LC-MS: 643.3 [M/2+H]⁺, 1285.6[M+H]⁺.

Step 5:

To about 150 mg of the relatively pure Compound 5 (oily substance) obtained by column chromatography, tetrahydrofuran (5 mL) and sodium hydroxide solution (10 mL, 2M) were added, the mixture was heated and refluxed, and reacted with temperature being maintained, tracked by TLC (MeOH:DCM=1:10). When the point of the raw material in the TLC disappeared, tetrahydrofuran was removed by rotary evaporation, and hydrochloric acid (6M) was added to adjust pH to 6-7. A white solid was precipitated, and cooled to 0-10° C., the temperature was maintained for 2 h, and the solid was subjected to suction filtering, and dried to afford 47.5 mg of Compound I-b, i.e., the bifunctional macrocyclic chelate.

LC-MS: 463.2[M/2+H]⁺, 925.4[M+H]⁺.

¹HNMR (600 MHz, D₂O): δ 8.26 (s, 4H), 8.01 (s, 4H), 7.45 (s, 4H), 7.14 (d, J=27.0 Hz, 4H), 4.61 (s, 8H), 4.43 (d, J=57.2 Hz, 8H), 4.14 (d, J=70.8 Hz, 8H), 2.19-1.99 (m, 6H).

### Example 3

The present example provides a bifunctional macrocyclic chelate (denoted as Dar-PEG), which has a structural formula as follows:

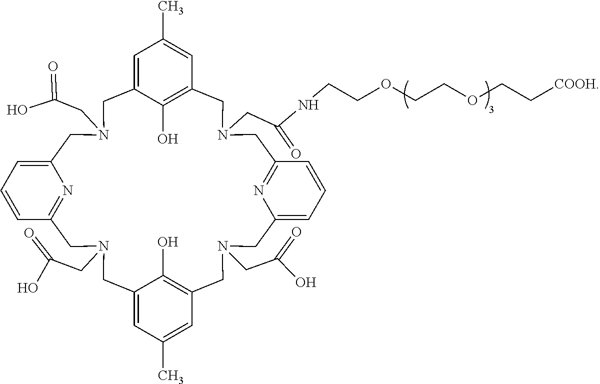

The bifunctional macrocyclic chelate of the present example was prepared by Fmoc solid phase synthesis reaction of the bifunctional macrocyclic chelate Dar (1.1 eq) in Example 1 and Compound G (1 eq) with N-hydroxy-7-azobenzotriazole (HOAt, 2.2 eq) and N,N′-diisopropyl carbodiimide (DIC, 2.2 eq) as condensing agents, followed by deprotection. In the above, the structural formula of Compound G is as follows:

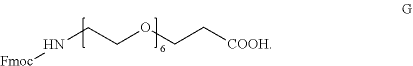

LC-MS: 509.9[M/2+H]⁺, 1018.7 [M+H]⁺.

### Example 4

The present example provides a bifunctional macrocyclic chelate (denoted as Dar-4PEG), which has a structural formula as follows:

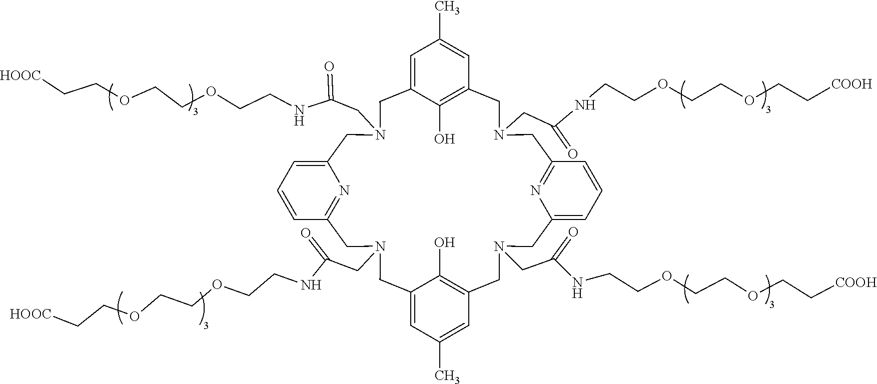

The bifunctional macrocyclic chelate in the present example was prepared by reaction of the bifunctional macrocyclic chelate Dar (1 eq) in Example 1 and Compound H (12 eq), with HOAt (1 eq) and DIC (1 eq) as condensing agents, followed by deprotection, in a yield of 51.37% and a purity of 98.10%. In the above, the structural formula of Compound H is as follows:

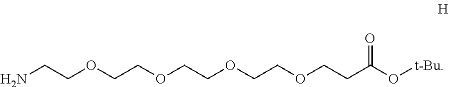 [SMILES: CC(C)(C)OC(=O)CCOCCOCCOCCOCCN]

LC-MS: 880.8 [M/2+H]⁺.

### Example 5

The present example provides a conjugate obtained by coupling Compound X and the bifunctional macrocyclic chelate in Example 1, wherein the structural formula of Compound X is as follows:

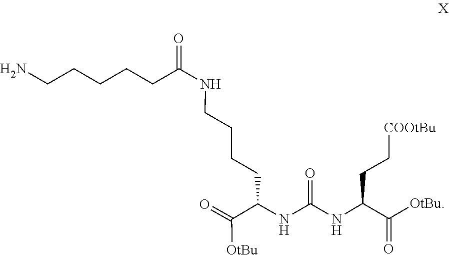

The experimental operations are as follows:

The sodium salt of Compound I-a in Example 1 (5 mg, 55 μmol, 1 eq) was stirred in anhydrous DMF (1 mL) at room temperature, HATU (8.37 mg, 22 μmol, 4 eq) was added, and after 1 h of stirring, Compound X (6.61 mg, 11 μmol, 2 eq) was added, pH was controlled to be 7-10, and the mixture reacted overnight, to afford an intermediate.

The intermediate was subjected to pre-treatment-high performance liquid chromatography purification (A: H₂O solution of 0.1% TFA, B: MeOH), followed by freeze drying to afford a colorless and viscous product (1.06 mg, 0.55 μmol, in a yield of 10%, and purity of 97.3%).

LC-MS: 969.1 [M/2+H]⁺.

The compound intermediate (1 mg) and HCl (200 μL, 6M) reacted at 60° C. for 10 min, and system changed from turbid to clear. After the reaction was completed, the reaction solution was neutralized to about pH 5, to afford the conjugate.

The conjugate was subjected to pre-treatment-high performance liquid chromatography purification (A: H₂O solution of 0.1% TFA, B: MeOH), followed by freeze drying to afford a colorless and viscous product (0.784 mg, 0.49 μmol, in a yield of 95%, and purity of 96.5%).

LC-MS: 800.7[M/2+H]⁺.

### Example 6

The present example provides a conjugate (denoted as Dar-PSMA-617), obtained by coupling Compound Y with the bifunctional macrocyclic chelate in Example 1, and synthesis was carried out according to the synthetic route as follows:

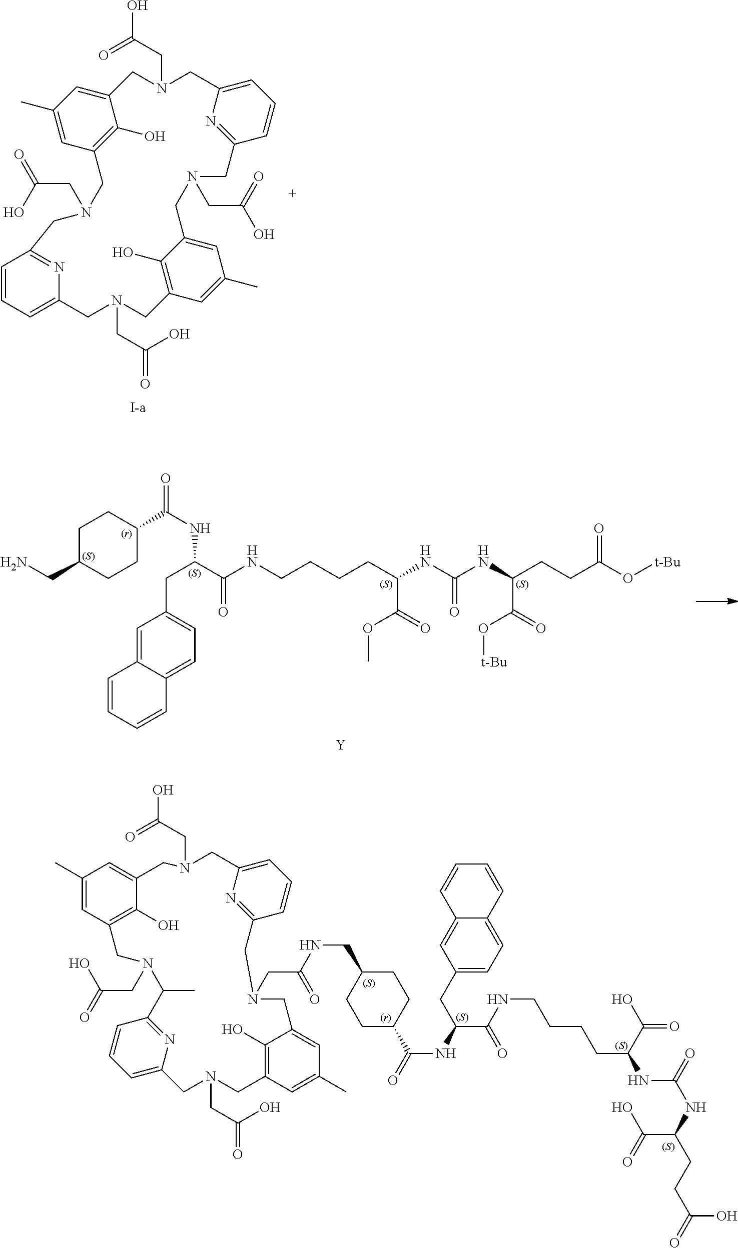

Compound I-a (78.0 mg, 101 μmol, 1.0 eq) in Example 1, HOAt (20.7 mg, 152 μmol, 1.5 eq), and DMF solution (6.0 mL) of DIC (19.2 mg, 152 μmol, 1.5 eq) were added dropwise to DMF (2.0 mL) solution of Compound Y (79.1 mg, 101 μmol, 1.0 eq) at room temperature, pH was around 8, and then reaction solution was stirred at 25° C. for 12 h.

Upon completion of reaction, the reaction solution was concentrated and dried at 50° C., and then treated with 7.0 mL of lysis solution (92.5% TFA/2.5% 3-MPA/2.5% tis/2.5% H₂O) for 2 h. The resultant mixture was precipitated with cold isopropyl ether (50 mL), subjected to centrifugation at 3000 rpm for 2 min, and washed twice with isopropyl ether. Crude product was dried in vacuo for 2 h. The resultant mixture was dissolved with ACN (3.0 mL) and diluted with H₂O (7.0 mL). PH was adjusted to 11-12 with 1 mol/L NaOH. Then the resultant mixture was stirred at 25° C. for 2 h. The mixture was freeze dried to afford a crude product.

The crude product was subjected to pre-treatment-high performance liquid chromatography purification (A: H₂O solution of 0.075% TFA, B: ACN), to afford a white solid Dar-PSMA-617 (8.1 mg, 5.48 μmol, in a yield of 5.41%, and purity of 95.27%).

LC-MS: 705.1 [M/2+H]⁺.

### Example 7

The present example provides a metal complex, obtained by complexing the conjugate prepared in Example 5 with ⁸⁹Zr, with operations as follows:

a conjugate solution (28 μL, 7 mg/mL) and ⁸⁹Zr (zirconium oxalate, 200 μCi, 50 μL) were fully mixed, and pH of reaction solution was adjusted and maintained at 5-6. Reaction was carried out at room temperature for 15 min, reaction solution was subjected to purification by C18 column, then the C18 column was washed with 10 mL of water, the C18 column was washed with 1 mL of 20% ethanol to afford ⁸⁹Zr-conjugate, and radiochemical purity was measured by HPLC to be 100%.

Identification:

preparation of standard: zirconium chloride (51 μg, 0.219 μmol, 1 eq), the conjugate prepared in Example 5 (350 μg, 0.219 μmol, 1 eq), and sodium acetate buffer (0.02 M, 20 μL) were mixed, and reacted at room temperature for 18 h, to afford a metal complex standard labeled with stable metal Zr.

LC-MS: 867.3[M/2+Na]⁺.

HPLC: standard UV peak Rt=11.468 min, radioactive peak Rt=11.589 min, corresponding to the peak position of the standard.

After the labeled product ⁸⁹Zr-conjugate was placed at room temperature for 48 h, radiochemical purity was 100%; after the labeled product ⁸⁹Zr-conjugate was placed in mouse serum and normal saline respectively, and incubated in 37° C. water bath incubator for 48 h, the radiochemical purity was 97% and 99% respectively, indicating that the labeled product ⁸⁹Zr-conjugate has good in vitro stability.

### Example 8

The present example provides a metal complex, obtained by complexing the conjugate prepared in Example 5 with ⁶⁸Ga, with operations as follows:

a conjugate solution (10 μL, 3 mg/mL) and a sodium acetate buffer of ⁶⁸GaCl₂ (1522 μCi, 50 μL) were fully mixed, and pH of reaction solution was adjusted and maintained at 6-7. Reaction was carried out at room temperature for 10 min, the reaction solution was subjected to purification by C18 column, then the C18 column was washed with 10 mL of water, the C18 column was washed with 1 mL of 20% ethanol to afford ⁶⁸Ga-conjugate, and radiochemical purity was measured to be 97.2%.

Identification:

preparation of standard: 50 μL of a conjugate solution (265 μg, 0.166 μmol) was taken, and pH was adjusted to be 5-6. 20 μL of gallium nitrate hydrate (20 μg, 0.073 μmol) dissolved in 0.02 M sodium acetate buffer with pH 7 was added, reaction was carried out at room temperature for 2 h, and the resultant mixture was filtered by filter membrane, to afford a metal complex standard labeled with stable metal Ga.

LC-MS: 834.3 [M/2+H]⁺.

HPLC: standard UV peak Rt=9.566 min, radioactive peak Rt=9.847 min, corresponding to the peak position of the standard.

After the labeled product ⁶⁸Ga-conjugate was placed at room temperature for 48 h, radiochemical purity was 97%; after the labeled product ⁶⁸Ga-conjugate was placed in mouse serum and normal saline respectively, and incubated in 37° C. water bath incubator for 48 h, the radiochemical purity was 96% and 97% respectively, indicating that the labeled product ⁶⁸Ga-conjugate has good in vitro stability.

### Example 9

The present example provides a metal complex (denoted as ⁸⁹Zr-Dar-PSMA-617), obtained by complexing the conjugate Dar-PSMA-617 prepared in Example 6 with ⁸⁹Zr, with operations as follows:

a conjugate solution (20 μL, 1 mg/mL) and ⁸⁹Zr (zirconium oxalate, 400 μCi, 50 μL) were fully mixed, and pH of reaction solution was adjusted and maintained at 6-7.

Reaction was carried out at room temperature for 15 min, the reaction solution was subjected to purification by C18 column, then the C18 column was washed with 10 mL of water, the C18 column was washed with 1 mL of 20% ethanol to afford ⁸⁹Zr-conjugate, and radiochemical purity was measured to be 98%.

Identification:

preparation of standard: a methanol solution (0.231 μmol, 50 μL, 6.5 mg/mL) of the conjugate in Example 6 was taken, pH was adjusted to be 5-6, a methanol solution of zirconium chloride (0.060 μmol, 14 μL, 1 mg/mL) was added, the mixture reacted at room temperature for 2 h, and was filtered by filter membrane, to afford a metal complex standard labeled with stable metal Zr.

LC-MS: 748.9 [M/2+H]⁺.

HPLC: standard UV peak Rt=10.548 min, radioactive peak Rt=10.892 min, corresponding to the peak position of the standard.

After the labeled product ⁸⁹Zr-conjugate was placed at room temperature for 48 h, radiochemical purity was 98%; after the labeled product ⁸⁹Zr-conjugate was placed in mouse serum and normal saline respectively, and incubated in 37° C. water bath incubator for 48 h, the radiochemical purity was 96% and 98% respectively, indicating that the labeled product ⁸⁹Zr-conjugate has good in vitro stability.

### Example 10

The present example provides a metal complex (denoted as ⁶⁸Ga-Dar-PSMA-617), obtained by complexing the conjugate Dar-PSMA-617 prepared in Example 6 with ⁶⁸Ga, with operations as follows:

a solution of the conjugate Dar-PSMA-617 (30 μL, 1 mg/mL) prepared in Example 6 and pre-treated sodium acetate buffer solution of ⁶⁸GaCl₂ (1522 μCi, 50 μL) were fully mixed, and pH of reaction solution was adjusted and maintained at 4-5. Reaction was carried out at room temperature for 10 min, the reaction solution was subjected to purification by C18 column, then the C18 column was washed with 10 mL of water, the C18 column was washed with 1 mL of 20% ethanol to afford ⁶⁸Ga-conjugate, and radiochemical purity was measured to be 97%.

Identification:

preparation of standard: a methanol solution (0.231 μmol, 50 μL, 6.5 mg/mL) of the conjugate Dar-PSMA-617 prepared in Example 6 was taken, pH was adjusted to be 5-6, a methanol solution (0.117 μmol, 32 μL, 1 mg/mL) of gallium nitrate was added, the mixture reacted at room temperature for 2 h, and was filtered by filter membrane, to afford a standard of metal complex standard labeled with stable metal Ga.

LC-MS: 738.7 [M/2+H]⁺.

HPLC: standard UV peak Rt=10.548 min, radioactive peak Rt=10.809 min, corresponding to the peak position of the standard. After the labeled product ⁶⁸Ga-conjugate was placed at room temperature for 48 h, radiochemical purity was 96%; after the labeled product ⁶⁸Ga-conjugate was placed in mouse serum and normal saline respectively, and incubated in 37° C. water bath incubator for 48 h, the radiochemical purity was 95% and 96% respectively, indicating that the labeled product ⁶⁸Ga-conjugate has good in vitro stability.

### Example 11

The present example provides a metal complex (denoted as ¹⁷⁷Lu-Dar-PSMA-617), obtained by complexing the conjugate Dar-PSMA-617 prepared in Example 6 with ¹⁷⁷Lu, with operations as follows:

a solution (50 μL, 0.05 mg) of the conjugate Dar-PSMA-617 prepared in Example 6 and ¹⁷⁷Lu (¹⁷⁷LuCl₃, 15 mCi) were fully mixed, and reacted at room temperature for 20 min. A reaction crude product was diluted to 10 mL and adsorbed by C18 column, the C18 column was washed with 10 mL of sterilized water for injection, then the C18 column was eluted with 0.5 mL of 80% ethanol, to afford 13 mCi of the product after elution, and the product was diluted to 4 mL with normal saline. Radiochemical purity was measured to be 100%.

Identification:

preparation of standard: lutetium chloride (62 μg, 0.219 μmol, 1 eq), a methanol solution (350 μg, 0.219 μmol, 1 eq) of the conjugate Dar-PSMA-617 prepared in Example 6, and a sodium acetate buffer (0.02 M, 20 μL) were mixed, and reacted at room temperature for 2 h, to afford a metal complex standard labeled with stable metal Lu.

LC-MS: 792.3 [M/2+H]⁺.

HPLC: standard UV peak Rt=9.012 min, radioactive peak Rt=9.238 min, corresponding to the peak position of the standard.

After the labeled product ¹⁷⁷Lu-conjugate was placed at room temperature for 48 h, radiochemical purity was 100%; after the labeled product ¹⁷⁷Lu-conjugate was placed in mouse serum and normal saline respectively, and incubated in 37° C. water bath incubator for 48 h, the radiochemical purity was 98% and 99% respectively, indicating that the labeled product ¹⁷⁷Lu-conjugate has good in vitro stability.

### Example 12

The present example provides a conjugate (denoted as Dar-Avastin), obtained by coupling Avastin with the bifunctional macrocyclic chelate Dar in Example 1, with operations as follows:

Avastin injection (450 μL, 11.25 mg) was diluted with NaOAc Buffer (3.2 mL, 0.1 M, pH 6.5), and the buffer was subjected to centrifugal replacement, to afford an Avastin solution (1.8 mL, 5.74 mg/mL).

84.1 mg of the sodium salt of Dar in Example 1 was dissolved in 3 mL of deionized water, and pH was adjusted with 0.1 M HCl to 5.5-6.0. 20.6 mg of Sulfo-NHS and 2.0 mg of EDC·HCl were added, and the mixture was shaken up, and left to stand at 4° C. for 30 min.

1.219 mL of Avastin solution was added, and the mixture was shaken up, pH of the solution was adjusted with 0.1 M Na₂CO₃ solution to 7.5-8.0, and the resultant mixture was shaken up, and left to stand at 4° C. overnight.

To the resulting Dar-Avastin coupling solution was added NaOAc Buffer (0.1 M, pH 6.5) for high-speed centrifugation (parameters of centrifugal machine: 7000 r, 12 min), filtrate collection tube was removed, and filtrate was poured out. The operations were repeated 2-4 times. 1.5 mL of Dar-Avastin solution was obtained at a concentration of 1.438 mg/mL.

### Example 13

The present example provides a metal complex (denoted as ⁸⁹Zr-Dar-Avastin), obtained by complexing the conjugate Dar-Avastin prepared in Example 12 with ⁸⁹Zr, with operations as follows:

⁸⁹Zr stock solution (zirconium oxalate, 60 μL, 1.22 mCi) was added to the Dar-Avastin solution prepared in Example 12. The mixture was shaken up, and added with 0.1 M Na₂CO₃ solution to adjust pH to 7.0, and the mixture reacted at room temperature overnight. Then, an appropriate amount of normal saline was added for high-speed centrifugation (parameters of centrifugal machine: 7000 r, 12 min), filtrate collection tube was removed, and filtrate was poured out. The operations were repeated 2-4 times. 270 μL ⁸⁹Zr-Dar-Avastin solution was obtained, with activity of 172 μCi, radiochemical purity measured by TLC was 100%, radiochemical purity measured by HPLC was 86.7%, and concentration was 4.5728 mg/mL.

### Example 14

The present example provides a conjugate (denoted as Dar-PEG-Avastin), with operations as follows:

antibody Avastin was coupled with Dar-PEG in Example 3, with operations as follows:

6.87 mg of Dar-PEG was dissolved in 1 mL of deionized water, and pH was adjusted with 0.1 M NaOH solution to 5.5-6.0. 1.5 mg of Sulfo-NHS was added, and the mixture was shaken up. EDC·HCl (9.6 μL, 13.5 mg/mL) was added, and the mixture was shaken up. The mixture was left to stand at 4° C. for 30 min. Then Avastin solution (400 μL, 2 mg) was added and shaken up, and pH was adjusted with 0.1 M NaOH solution to 7.5-8.0, the resultant mixture was shaken up, and left to stand at 4° C. overnight.

To the resulting Dar-PEG-Avastin coupling solution, an appropriate amount of NaOAc Buffer (0.1 M, pH 6.5) was added for high-speed centrifugation (parameters of centrifugal machine: 7000 r, 12-15 min), filtrate collection tube was removed, and filtrate was poured out. The above operations were repeated 2-4 times. Finally, 400 μL of NaOAc Buffer (0.1 M, pH 6.5) was added for dilution to afford a Dar-PEG-Avastin solution, at a concentration of 1.49 mg/mL.

### Example 15

The present example provides a metal complex (denoted as ¹⁷⁷Lu-Dar-PEG-Avastin), obtained by complexing the conjugate Dar-PEG-Avastin prepared in Example 14 with ¹⁷⁷Lu, with operations as follows:

550 μL of the Dar-PEG-Avastin solution (1.49 mg/mL) prepared in Example 14 was diluted with 500 μL of 0.1 M NaOAc Buffer.

To NaOAc Buffer (400 μL, 0.2 M, pH 4.64) was added ¹⁷⁷Lu stock solution (35 μL, 21.4 mCi), and the mixture was shaken up, to afford ¹⁷⁷Lu solution, which was measured by TLC, showing almost no colloid generation.

To the Dar-PEG-Avastin solution was added 215 μL of the ¹⁷⁷Lu solution, and the mixture was shaken up, to measure pH to be 6.0. Reaction was carried out for 30 min at room temperature, sample was detected by TLC, and when the labelling rate was shown to be 100%, the reaction was stopped.

The reaction solution was subjected to purification by PD-10 column, and was eluted with normal saline, 500 μL×5, and the second tube was collected to afford 500 μL, 2.05 mCi, and a sample was measured by HPLC, and radiochemical purity was 100%.

### Example 16

The present example provides a conjugate, with operations as follows:

Dar-NCS was formed using the bifunctional macrocyclic chelate Dar in Example 1, and synthesis was carried out according to the synthetic route as follows:

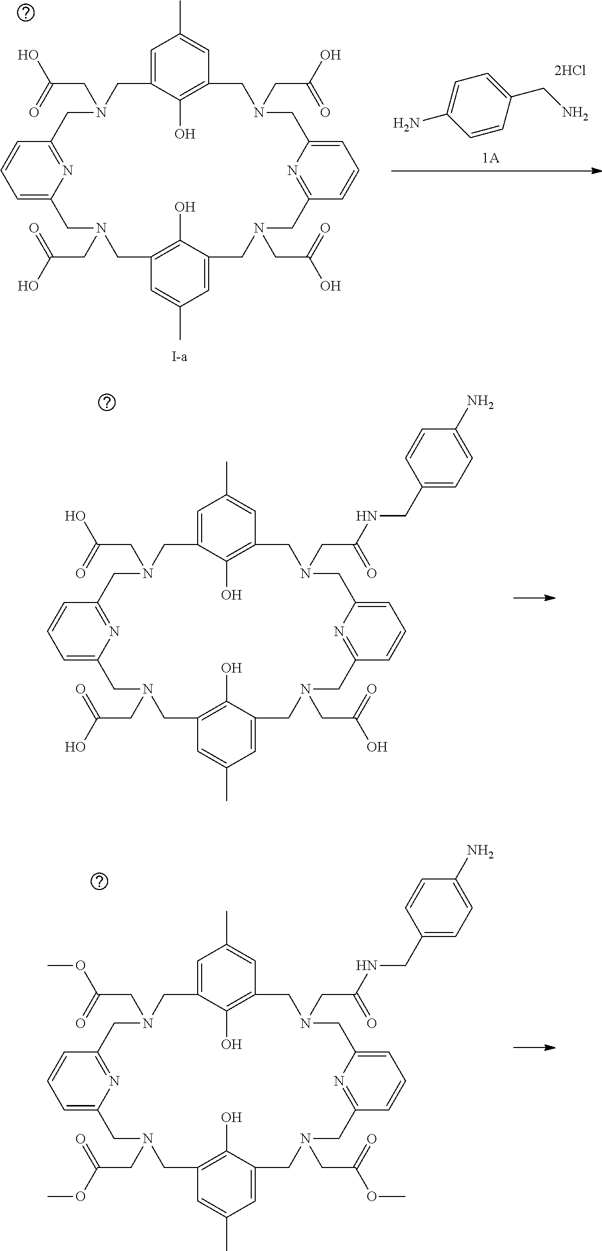

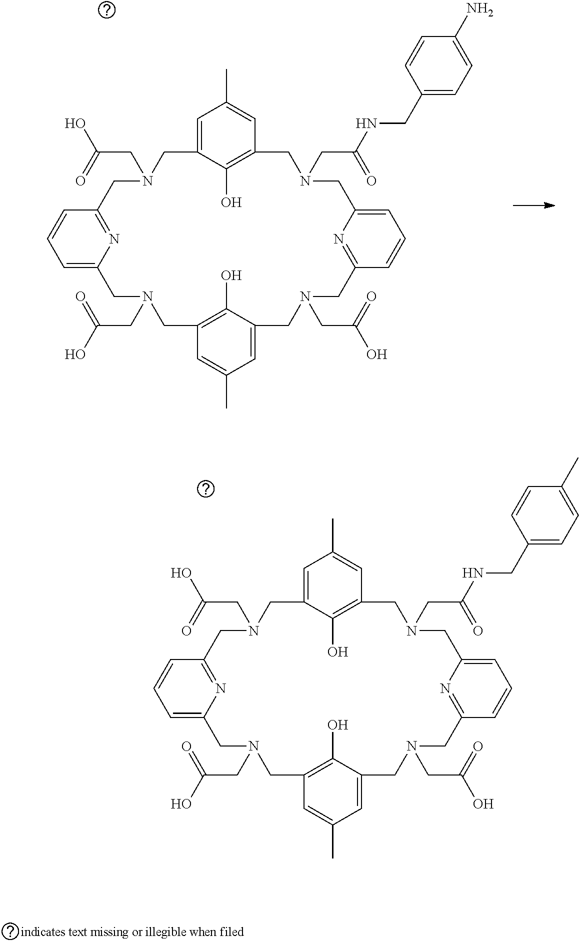 [SMILES: Cc1cc2c(O)c(c1)CN(CC(=O)O)Cc1cccc(n1)CN(CC(=O)NCc1ccc(N)cc1)Cc1cc(C)cc(c1O)CN(CC(=O)O)Cc1cccc(n1)CN(CC(=O)O)C2 ; Cc1ccc(CNC(=O)CN2Cc3cccc(n3)CN(CC(=O)O)Cc3cc(C)cc(c3O)CN(CC(=O)O)Cc3cccc(n3)CN(CC(=O)O)Cc3cc(C)cc(c3O)C2)cc1]

The experimental operations are as follows:

Step 1:

To a DMF (15.0 mL) suspension of Compound I-a (i.e., Dar) (260 mg, 269 μmol, purity 80%, 1.00 eq) and Compound 1A (42.1 mg, 216 μmol, 39.0 μL, 0.80 eq, 2HCl) was added TEA (410 mg, 4.05 mmol, 563 μL, 15.0 eq), and the mixture was stirred until reactants were completely dissolved. Subsequently, a DMF (5.00 mL) solution of HBTU (102 mg, 270 μmol, 1.00 eq) was added in batches at 20° C. within 1 h. The mixture was stirred at 20° C. for 14 h to afford Intermediate I.

LC-MS: 875.1 [M+H]⁺.

Step 2:

Intermediate I crude product (300 mg, 274 μmol, 1.00 eq, 6HCl) was dissolved in methanol (20.0 mL), solution was cooled to −5° C., then thionyl dichloride (6.56 g, 55.1 mmol, 4.00 mL, 201 eq) was added, and reaction mixture was stirred at 25° C. for 1 h and then heated at 45° C. for 1 h. The completion of the reaction was determined by LC-MS, reaction mixture was concentrated in vacuo and then dissolved in water (5 mL), a white solid was precipitated by alkalization with sodium bicarbonate aqueous solution, the white solid was extracted with ethyl acetate (5 mL), the organic phase was separated and concentrated in vacuo, the resulting residue was purified by preparative HPLC, followed by freeze drying to afford Intermediate II (80 mg, 87.24 μmol) of the white solid, in a yield of 31.8%, and purity of 100%.

LC-MS: 917.5 [M+H]⁺, 459.4 [M/2+H]⁺.

Step 3:

Intermediate II (30.0 mg, 32.7 μmol, 1.00 eq) was added to a THF aqueous solution (50%, 4 mL) and dissolved, then lithium hydroxide monohydrate (27.5 mg, 654 μmol, 20.0 eq) was added in batches, the mixture was stirred at 25° C. for 14 h. THF was removed and the residue was acidified with 1 M hydrochloric acid aqueous solution to pH=5. The acidified aqueous solution was purified by preparative HPLC to afford the intermediate I (15 mg, 13.7 μmol, 6HCl), a white solid, in a yield of 41.9%.

LC-MS: 875.4 [M+H]⁺.

Step 4:

To a dichloromethane solution of the intermediate I (15.0 mg, 13.7 μmol, 1.00 eq, 6HCl) was added carbon disulfide (15.7 mg, 137 μmol, 10.5 μL, 10.0 eq), and the mixture was stirred at 15° C. for 4 h. Reaction mixture was extracted with dichloromethane (5.00 mL×3) to remove excess carbon disulfide, and the aqueous layer was directly freeze dried to afford Dar-NCS (5 mg, 4.22 μmol, 6HCl), a white solid, in a yield of 30.7%, and purity of 95.8%.

LC-MS: 917.5 [M+H]⁺, 459.4 [M/2+H]⁺.

¹HNMR (400 MHz, D₂O):δ: 7.88-7.78 (m, 2H), 7.45-7.37 (m, 3H), 7.34-7.28 (m, 1H), 7.25-7.15 (m, 4H), 7.10 (d, J=8.4 Hz, 2H), 6.92 (d, J=8.4 Hz, 2H), 4.61-4.37 (m, 16H), 4.13 (s, 2H), 3.98 (s, 2H), 3.91-3.79 (m, 6H), 2.15 (s, 6H).

Antibody Avastin was coupled with Dar-NCS, with operations as follows:

Avastin injection (450 μL, 11.25 mg) was diluted with NaOAc Buffer (3.2 mL, 0.1 M, pH 6.5), and the buffer was subjected to centrifugal replacement, to afford an Avastin solution (1.8 mL, 5.74 mg/mL). Avastin solution (350 μL, 5.74 mg/mL) was added to Dar-NCS solution (350 μL, 0.01224 mg/mL) and the mixture was gently shaken up. The solution was slowly adjusted with 0.1 M Na₂CO₃ solution to pH 8.5, and the resultant mixture was gently shaken up. Then centrifuge tube was placed in a 37° C. heating plate and heated for 1 h, and then left to cool overnight. The resulting Dar-NCS-Avastin coupling solution was subjected to centrifugation with deionized water (parameters of centrifugal machine: 6500 r, 15 min). The operations were repeated 2-4 times.

### Example 17

The present example provides a conjugate (denoted as Dar-KN035), obtained by coupling KN035 with the bifunctional macrocyclic chelate Dar in Example 1, with operations as follows:

KN035 injection (450 μL, 11.25 mg) was diluted with NaOAc Buffer (3.2 mL, 0.1 M, pH 6.5), and the buffer was subjected to centrifugal replacement, to afford a KN035 solution (1.8 mL, 5.74 mg/mL).

84.1 mg of the sodium salt of Dar in Example 1 was dissolved in 3 mL of deionized water, and the mixture was adjusted with 0.1 M HCl to pH 5.5-6.0. 20.6 mg of Sulfo-NHS and 2.0 mg of EDC. HCl were added, and the mixture was shaken up, and left to stand at 4° C. for 30 min.

1.219 mL of KN035 solution was added, and the mixture was shaken up, the solution was adjusted with 0.1 M Na₂CO₃ solution to pH 7.5-8.0, and the resultant mixture was shaken up, and left to stand at 4° C. overnight.

To the resulting Dar-KN035 coupling solution was added NaOAc Buffer (0.1 M, pH 6.5) for high-speed centrifugation (parameters of centrifugal machine: 7000 r, 12 min), filtrate collection tube was removed, and filtrate was poured out. The operations were repeated 2-4 times. 1.5 mL of a Dar-KN035 solution was obtained, at a concentration of 1.438 mg/mL.

### Example 18

The present example provides a metal complex (denoted as ⁸⁹Zr-Dar-KN035), obtained by complexing the conjugate Dar-KN035 prepared in Example 17 with ⁸⁹Zr, with operations as follows:

⁸⁹Zr stock solution (zirconium oxalate, 60 μL, 1.22 mCi) was added to the Dar-KN035 solution prepared in Example 17, the mixture was shaken up, and added with 0.1 M Na₂CO₃ solution to adjust pH to 7.0, and the mixture reacted at room temperature overnight. Then, an appropriate amount of normal saline was added for high-speed centrifugation (parameters of centrifugal machine: 7000 r, 12 min), filtrate collection tube was removed, and filtrate was poured out. The operations were repeated 2-4 times. 270 μL of ⁸⁹Zr-Dar-KN035 solution was obtained, with activity of 172 μCi, radiochemical purity measured by HPLC was 96%, and concentration was 4.5728 mg/mL.

### Experimental Example 1: PET Imaging

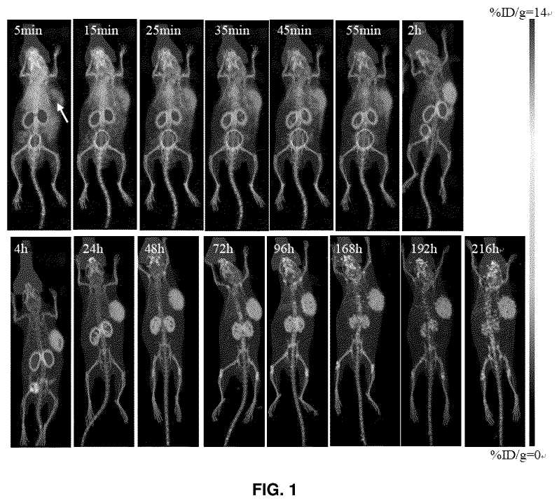

Method: LNCap tumor-bearing mice were injected with the complex ⁸⁹Zr-Dar-PSMA-617 (200 μCi) prepared in Example 9 through tail vein, while dynamic scan was performed for 60 min during the administration, and static whole body scan was performed for 10 min at different time points during 2-216 h after the administration. Refer to FIG. 1 for the mouse PET scanning image.

Conclusion: (1) after a single intravenous administration of ⁸⁹Zr-Dar-PSMA-617 to LNCap tumor-bearing mice, radioactive substances were mainly distributed in the bladder and kidneys, and the radioactive uptake value thereof rapidly decreased within 2 h; the radioactive substances were less distributed in other tissues (lung, liver, heart, etc.); and (2) the radioactive uptake value of tumor peaked at 4 h, and subsequently gradually declined over time. Animal trial studies show that ⁸⁹Zr-Dar-PSMA-617 has good diagnostic effects, and provides possibilities for development of therapeutic medicines and medication guidance.

### Experimental Example 2: PET Imaging

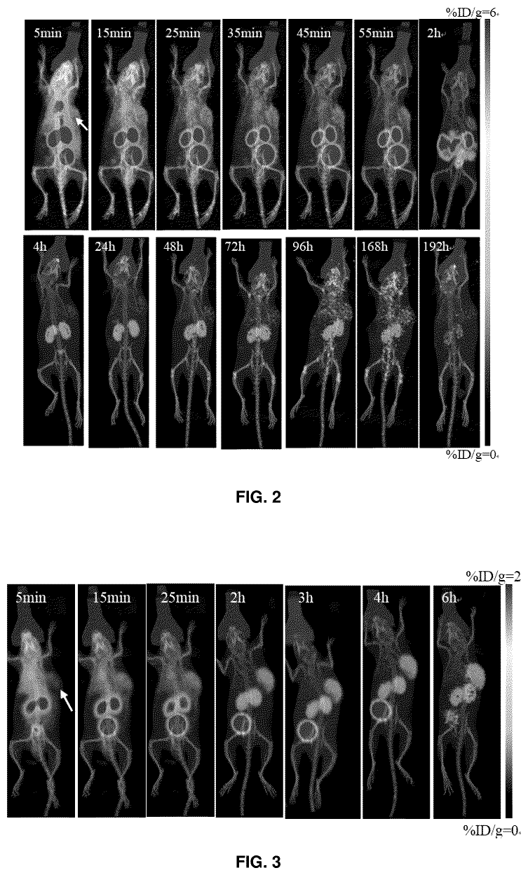

Method: LNCap tumor-bearing mice were injected with the complex (200 μCi) prepared in Example 7 through tail vein, dynamic scan was performed for 60 min during the administration, and the static whole body scan was performed for 10 min at different time points of 2-192 h after the administration. Refer to FIG. 2 for the mouse PET scanning image.

Conclusion: (1) after a single intravenous administration of the complex prepared in Example 7 to LNCap tumor-bearing mice, radioactive substances were mainly distributed in the bladder and kidneys, and the radioactive uptake value thereof rapidly decreased within 2 h; the radioactive substances were less distributed in other tissues (lung, liver, heart, etc.); and (2) the radioactive uptake value of tumor peaked at 15 min, and subsequently gradually declined over time. Animal trial studies show that the present product has good clinical application prospect.

### Experimental Example 3: PET Imaging

Method: LNCap tumor-bearing mice were injected with the complex ⁶⁸Ga-Dar-PSMA-617 (200 μCi) prepared in Example 10 through tail vein, dynamic scan was performed for 60 min during the administration, and the static whole body scan was performed for 10 min at different time points of 2-6 h after the administration. Refer to FIG. 3 for the mouse PET scanning image.

Conclusion: significant tumor uptake was seen 1 h after the administration, with relatively low uptake in other non-target organs. ⁶⁸Ga-Dar-PSMA-617 was mainly excreted through the kidneys. It can be seen that the present product has relatively good stability and targeting property in vivo, and is expected to become a novel targeted diagnostic reagent.

### Experimental Example 4: Pharmacokinetic Study

### Comparative Example 1

The preparation process of metal complex ⁸⁹Zr-Dar is as follows:

5 mCi of ⁸⁹Zr (zirconium oxalate) and Dar (50 μL, 0.05 mg) were mixed, reacted at normal temperature for 2 h; the reaction crude product obtained was diluted to 10 mL and adsorbed by C18 column, the C18 column was washed with 10 mL of sterilized water for injection; and then the C18 column was eluted with 0.5 mL of 80% ethanol, and 4 mCi of ⁸⁹Zr-Dar obtained from the elution was diluted to 4 mL with normal saline.

Method: SD rats were used for pharmacokinetic experiments, and respectively injected intravenously with approximately 80 μCi of ⁸⁹Zr-Dar and 80 μCi of ⁸⁹Zr-Dar-PSMA-617 prepared in Example 9, with 12 male rats in each group. About 0.1-0.2 mL of blood was collected from jugular vein plexus of the experimental animals at 5 min, 15 min, 30 min, 1 h, 2 h, 3 h, 4 h, 5 h, 6 h, 8 h, 12 h, 24 h, 48 h, 72 h, 96 h, 120 h, 144 h, and 168 h after the administration, and the rats were weighed for subsequent assay. Blood was collected from 6 males in each group at each time point, and 12 animals were cross-sampled. Each sample was subjected to gamma counting for 30 s.

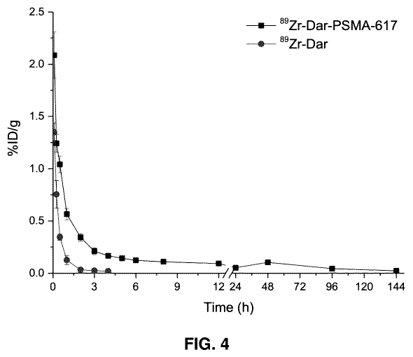

Results are as follows:
(1) animal clinical observation: there was no abnormality in the conditions of the animals during the experiment; and
(2) the drug concentration-time curve is shown in FIG. 4, and it can be seen that half-life period of ⁸⁹Zr-Dar in blood is 12.6 min, and half-life period of ⁸⁹Zr-Dar-PSMA-617 in blood is 4.57 h.

### Experimental Example 5

### Comparative Example 2

The preparation process of metal complex ¹⁷⁷Lu-Dar is as follows:

5 mCi of ¹⁷⁷Lu (¹⁷⁷LuCl₃) and Dar (50 μL, 0.05 mg) were mixed and reacted at normal temperature for 2 h; the reaction crude product obtained was diluted to 10 mL and adsorbed by C18 column, the C18 column was washed with 10 mL of sterilized water for injection; and then the C18 column was eluted with 0.5 mL of 80% ethanol, and 4 mCi of ⁸⁹Zr-Dar obtained from the elution was diluted to 4 mL with normal saline.

### Comparative Example 3

A preparation process of metal complex ¹⁷⁷Lu-DOTA is as follows:

5 mCi of ¹⁷⁷Lu (¹⁷⁷LuCl₃) and DOTA (50 μL, 0.05 mg) were mixed, reacted at 90° C. for 1 h; the reaction crude product obtained was diluted to 10 mL and adsorbed by C18 column, the C18 column was washed with 10 mL of sterilized water for injection; and then the C18 column was eluted with 0.5 mL of 80% ethanol, and 4 mCi of ¹⁷⁷Lu-DOTA obtained from the elution was diluted to 4 mL with normal saline.

### Comparative Example 4

The preparation process of metal complex ¹⁷⁷Lu-DOTA-PSMA-617 is as follows:

5 mCi of ¹⁷⁷Lu (¹⁷⁷LuCl₃) and DOTA-PSMA-617 (50 μL, 0.05 mg) were mixed, reacted at 90° C. for 1 h; the reaction crude product obtained was diluted to 10 mL and adsorbed by C18 column, the C18 column was washed with 10 mL of sterilized water for injection; and then the C18 column was eluted with 0.5 mL of 80% ethanol, and 4 mCi of ¹⁷⁷Lu-DOTA-PSMA-617 obtained from the elution was diluted to 4 mL with normal saline.

### Comparative Example 5

Refer to Linker Modification Strategies To Control the Prostate-Specific Membrane Antigen (PSMA)-Targeting and Pharmacokinetic Properties of DOTA-Conjugated PSMA Inhibitors, Benešova et al, J. Med. Chem. 2016, 59, 1761-1775 for the preparation process of metal complex DOTA-PSMA-617

Experimental Preparation:

LNCap cells were cultured to logarithmic growth phase. After digestion with pancreatin, the resultant mixture was adjusted with complete medium into a cell suspension with a cell density of 1×10⁶-2×10⁶ cells/mL. Cells were inoculated in 24-well plate with 0.5-1.0 mL cells per well and the number of 1×10⁵ cells per well (inoculated 2 d in advance for cell experiment). ¹⁷⁷Lu-Dar, ¹⁷⁷Lu-Dar-PSMA-617, ¹⁷⁷Lu-DOTA, and ¹⁷⁷Lu-DOTA-PSMA-617 were respectively diluted with a basic medium (with 1% HSA added) to 10 μCi/mL for subsequent use.

Unlabeled ligands Dar, DOTA, Dar-PSMA-617, and DOTA-PSMA-617 were respectively formulated to be 10 μg/mL and 25 μg/mL with basic medium (with 1% HSA added) for subsequent use.

Experiment A:

Cells were incubated in 37° C. incubator for 1 h, 4 h, and 24 h.

Uptake experiment: blocking groups were set in cell uptake experiment, and unlabeled ligands were added in an amount of 200 times that of labeled subjects. After incubation was completed, the cell suspension was collected, followed by centrifugation at 1500 rpm for 5 min, cell pellet was collected, the supernatant was removed, the cell pellet was washed once with 0.5 mL of pre-cooled PBS, and the resultant cell pellet was collected.

Internalization experiment: after incubation was completed, the cell suspension was collected, 0.5 mL of pre-cooled pickling solution was added, after incubation for 1 min at room temperature, the pickling solution was removed; and the resultant mixture was washed once with 0.5 mL of PBS, and cell pellet was collected.

Efflux experiment: after the incubation was completed, the cell suspension was collected, washed once with 0.5 mL of complete medium, added with 0.5 mL of complete medium, after resuspension, the resultant mixture was placed into original wells, incubated at 37° C. for 24 h, and then the supernatant was collected, and washed once with 0.5 mL of PBS. Cell pellet was collected after centrifugation of the supernatant and PBS washing solution; and 0.5 mL of 1 M lysis solution was utilized to digest and lyse cells in the original wells, and combined with the cell pellet.

Experiment B:

Cells were incubated in 37° C. incubator for 4 h and 24 h.

Uptake experiment: blocking groups were set in cell uptake experiment, and unlabeled ligands were added in an amount of 600 times that of labeled subjects. After incubation was completed, the cell suspension was collected, upon followed by centrifugation at 1500 rpm for 5 min, cell pellet was collected, the supernatant was removed, the cell pellet was washed once with 0.5 mL of pre-cooled PBS, and the cell pellet was collected.

Internalization experiment: after incubation was completed, the cell suspension was collected, 0.5 mL of pre-cooled pickling solution was added, after incubation for 1 min at room temperature, the pre-cooled pickling solution was removed; the resultant mixture was washed once with 0.5 mL of PBS, and cell pellet was collected.

Efflux experiment: after the incubation was completed, the cell suspension was collected, washed once with 0.5 mL of complete medium, added with 0.5 mL of complete medium, after resuspension, the resultant mixture was placed into original wells, incubated at 37° C. for 24 h, and then the supernatant was collected, and washed once with 0.5 mL of PBS. Cell pellet was collected after centrifugation of the supernatant and PBS washing; and 0.5 mL of 1 M lysis solution was utilized to digest and lyse cells in the original wells.

Data Processing:

Cell pellet was collected for gamma counting. Both the blank and standard tubes were assayed simultaneously in each test.

Cell binding rate=CPM of the cell lysate/CPM of the standard tube×100%

Cell internalization rate=CPM of the cell lysate after acid pickling/CPM of the uptake experiment cell lysate×100%

Cell efflux rate=CPM of the efflux experiment cell lysate/CPM of the uptake experiment cell lysate×100%

Experimental Results:

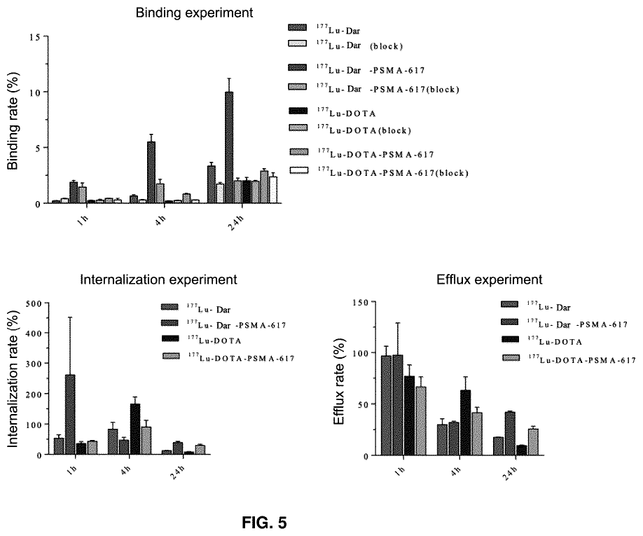

Experiment A: according to FIG. 5 it can be seen that in the uptake experiment, uptake rates of ¹⁷⁷Lu-subjects and LNCap cells increase gradually over time. Uptake rates of ¹⁷⁷Lu-Dar and LNCap cells at 1 h, 4 h, and 24 h are 0.21%, 0.65%, and 3.35% respectively; and uptake rates of the blocking groups are 0.40%, 0.28%, and 1.72% respectively. Uptake rates of ¹⁷⁷Lu-Dar-PSMA-617 and LNCap cells at 1 h, 4 h, and 24 h are 1.88%, 5.51%, and 9.96%; uptake rates of the blocking groups are 1.45%, 1.73%, and 2.01% respectively. Uptake rates of ¹⁷⁷Lu-DOTA and LNCap cells at 1 h, 4 h, and 24 h are 0.22%, 0.20%, and 2.01% respectively; uptake rates of the blocking groups are 0.26%, 0.24%, and 1.97% respectively. Uptake rates of ¹⁷⁷Lu-DOTA-PSMA-617 and LNCap cells at 1 h, 4 h, and 24 h are 0.45%, 0.82%, and 2.88% respectively; and uptake rates of the blocking groups are 0.29%, 0.28%, and 2.36%.

Internalization experiment: internalization rates of ¹⁷⁷Lu-Dar and LNCap cells at 1 h, 4 h, and 24 h are 53.31%, 83.64%, and 12.25% respectively. Internalization rates of ¹⁷⁷Lu-Dar-PSMA-617 and LNCap cells at 1 h, 4 h, and 24 h are 261.51%, 47.07%, and 38.92%. Internalization rates of ¹⁷⁷Lu-DOTA and LNCap cells at 1 h, 4 h, and 24 h are 35.46%, 165.46%, and 8.33%. Internalization rates of ¹⁷⁷Lu-DOTA-PSMA-617 and LNCap cells at 1 h, 4 h, and 24 h are 43.33%, 90.03%, and 29.70%.

Efflux experiment: efflux rates of ¹⁷⁷Lu-Dar and LNCap cells at 1 h, 4 h, and 24 h are 96.57%, 29.87%, and 17.49% respectively. Efflux rates of ¹⁷⁷Lu-Dar-PSMA-617 and LNCap cells at 1 h, 4 h, and 24 h are 97.32%, 31.93%, and 41.87% respectively. Efflux rates of ¹⁷⁷Lu-DOTA and LNCap cells at 1 h, 4 h, and 24 h are 76.83%, 63.22%, and 9.38% respectively. Efflux rates of ¹⁷⁷Lu-DOTA-PSMA-617 and LNCap cells at 1 h, 4 h, and 24 h are 66.48%, 41.44%, and 25.75% respectively.

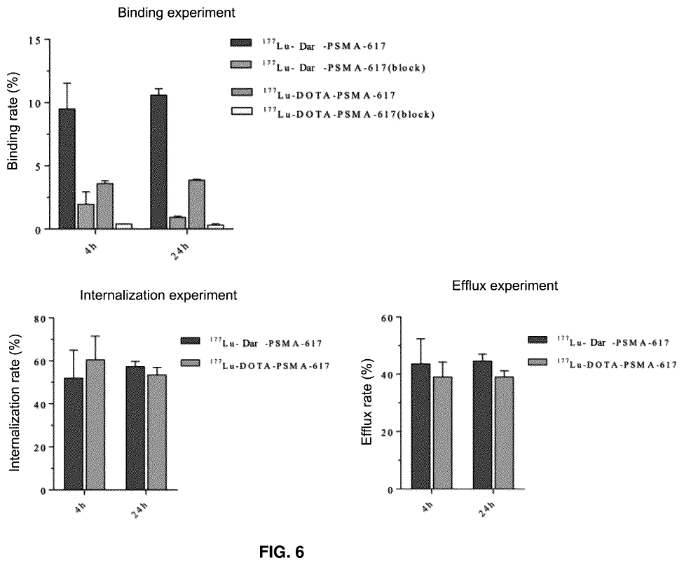

Experiment B: it can be seen according to FIG. 6 that in the uptake experiment, uptake rates of ¹⁷⁷Lu-Dar-PSMA-617 and LNCap cells at 4 h and 24 h are 9.50% and 10.59% respectively; and uptake rates of the blocking groups are 1.95% and 0.92% respectively. Uptake rates of ¹⁷⁷Lu-DOTA-PSMA-617 and LNCap cells at 4 h and 24 h are 3.60% and 3.87% respectively; uptake rates of the blocking groups are 0.39% and 0.31% respectively. Uptake rates of ¹⁷⁷Lu-Dar-PSMA-617 and LNCap cells are higher than that of ¹⁷⁷Lu-DOTA-PSMA-617, and there is no significant difference between the two time points.

Internalization experiment: internalization rates of ¹⁷⁷Lu-Dar-PSMA-617 and LNCap cells at 4 h and 24 h are 51.92% and 57.22% respectively. Internalization rates of ¹⁷⁷Lu-DOTA-PSMA-617 and LNCap cells at 4 h and 24 h are 60.43% and 53.41% respectively. Internalization rates of ¹⁷⁷Lu-Dar-PSMA-617 and LNCap cells increased over time, while internalization rate of ¹⁷⁷Lu-DOTA-PSMA-617 decreased over time.

Efflux experiment: efflux rates of ¹⁷⁷Lu-Dar-PSMA-617 and LNCap cells at 4 h and 24 h are 43.59% and 44.60% respectively. Efflux rates of ¹⁷⁷Lu-DOTA-PSMA-617 and LNCap cells at 4 h and 24 h are 39.01% and 39.05% respectively. Efflux rates of ¹⁷⁷Lu-Dar-PSMA-617 and LNCap cells are comparable with that of ¹⁷⁷Lu-DOTA-PSMA-617.

### Experimental Example 6: ¹⁷⁷Lu-Dar-PSMA-617 Treatment Study

LNCap tumor-bearing mice animal models were used.

Grouping:

Experimental Groups:
(A)¹⁷⁷Lu-Dar-PSMA-617 low dosage 0.25 mCi (n=7);
(B)¹⁷⁷Lu-Dar-PSMA-617 medium dosage 0.5 mCi (n=10);
(C)¹⁷⁷Lu-Dar-PSMA-617 high dosage 1 mCi (n=10).

Positive Control Groups:
(D)¹⁷⁷Lu-DOTA-PSMA-617 0.5 mCi (n=7);
(E)¹⁷⁷Lu-DOTA-PSMA-617 1 mCi (n=10).

Negative Control Groups:
(F) ¹⁷⁷LuCl₃ 1 mCi (n=10);
(G) normal saline (n=10)

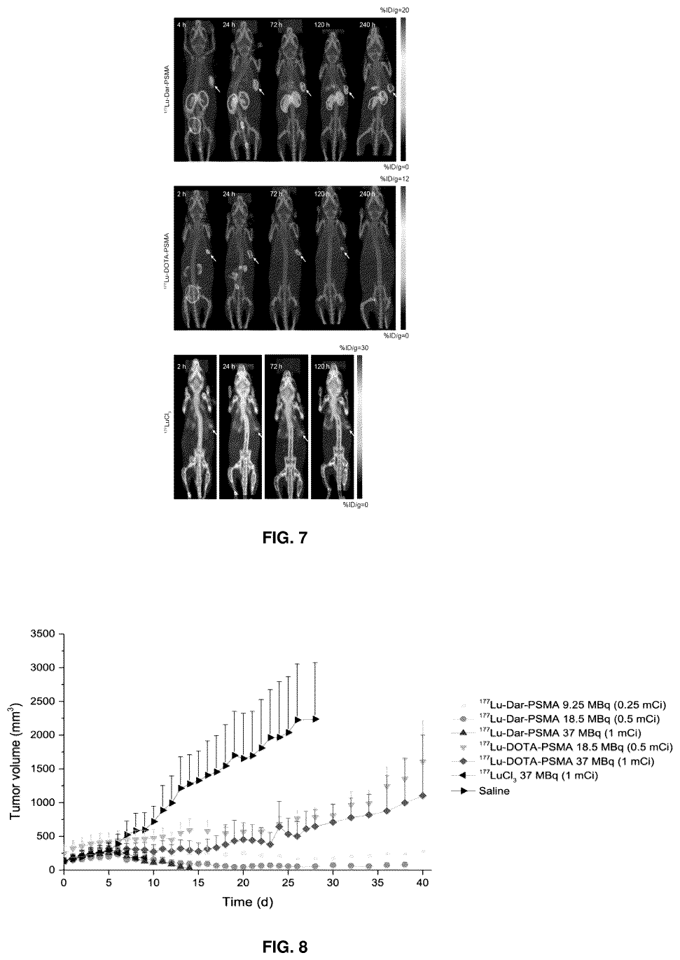

Method: the day of administration was recorded as D-0, and body weight and tumor size were measured every day or every other day. After administration of ¹⁷⁷Lu-Dar-PSMA-617 (Group C), SPECT scan was performed at 4 h, 24 h, 72 h, 120 h, and 240 h after the administration; the control groups ¹⁷⁷Lu-DOTA-PSMA-617 (Group E) and ¹⁷⁷LuCl₃ (Group F) were respectively subjected to SPECT scan at 2 h, 24 h, 72 h, and 120 h (240 h) (refer to FIG. 7, group C, group E, and group F in sequence from top to bottom). The animals were kept stationary, and CT scan was completed before/after the SPECT scan. Prior to the scan, the animals were subjected to respiratory anesthesia with isoflurane through an anesthesia machine, and the animals having undergone anesthesia induction were placed on a SPECT/CT bed. During the scan, the animals continuously inhaled isoflurane to maintain anesthesia effect. Each bed was statically scanned for 10-30 min and scan time was recorded.

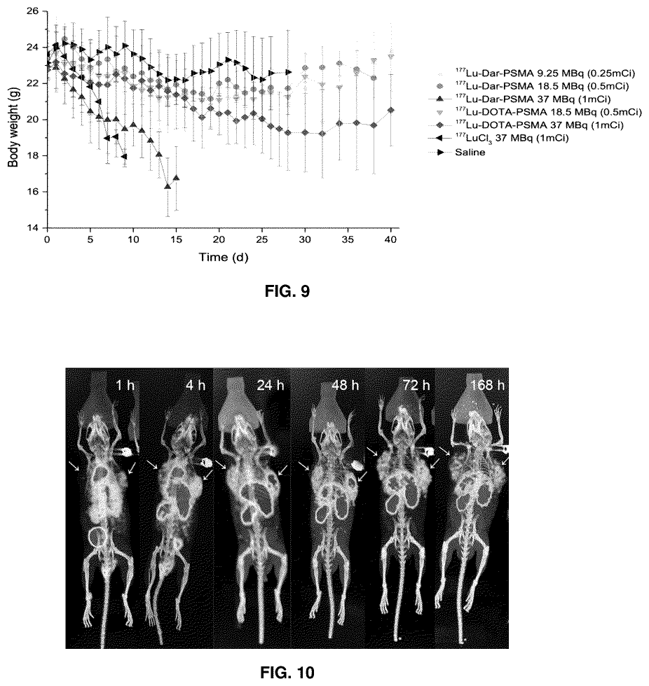

Experimental Results:
(1) animal clinical observation: there is no abnormality in the state of the animals in the ¹⁷⁷Lu-Dar-PSMA-617 low dosage group (Group A) during the experiment. There is no abnormality in the state of the animals in the ¹⁷⁷Lu-DOTA-PSMA-617 medium dosage group (Group D) during the experiment. During the experiment of the ¹⁷⁷LuCl₃ group (Group F), starting from D-5, some of the animals show phenomena of weight loss and arched back, and all the animals show the phenomena of weight loss and arched back at the later stage of the experiment. It is found in gross anatomy of dead mice in Group F the phenomena of skeletal darkening and blood stasis of bone marrow, proving that negative control of radioactive reagent ¹⁷⁷LuCl₃ produces severe adverse effects.
(2) It can be seen from FIG. 8 that compared with the control normal saline group (Group G), the tumor size of the animals in experimental groups A-C and positive control groups D and E grow slowly, and even at later stages of the experiment, there appears the trend that the tumor gradually decreases (¹⁷⁷Lu-Dar-PSMA-617 medium dosage group (Group B)), which indicates that the ¹⁷⁷Lu-Dar-PSMA-617 in the present disclosure possesses a significant inhibitory effect on the growth of LNCap tumor, and the effect is remarkably superior to that of positive drug ¹⁷⁷Lu-DOTA-PSMA-617 in the prior art. It can be seen from FIG. 9 that the body weight gradually decreases, wherein Group C and Group F decrease rapidly. Adverse effects such as rapid weight loss, diarrhea, and hair loss are not observed in other groups.

### Experimental Example 7: ⁸⁹Zr-Dar-KN035 Imaging Study

Method: MC38/MC38-hPDL1 bilateral tumor-bearing mice were injected with the ⁸⁹Zr-Dar-KN035 (100 μCi) through tail vein, wherein MC38 was a tumor low-expressing PDL1, and MC38-hPDL1 was a tumor high-expressing PDL1. Dynamic scan was performed for 60 min during the administration, and static whole body scan was performed for 10 min at different time points of 1-168 h after the administration. A mouse PET scanning image is shown in FIG. 10, with MC38 on the left and MC38-hPDL1 on the right.

It can be seen from FIG. 10 that ⁸⁹Zr-Dar-KN035 was significantly taken up in tissues high-expressing tumor and was almost not taken up in tissues low-expressing tumor, indicating that ⁸⁹Zr-Dar-KN035 has good tumor selectivity, and sufficiently proving that the ⁸⁹Zr-Dar-KN035 in the present disclosure has great potential for being developed into high-efficiency low-toxicity anti-tumor drugs.

The foregoing descriptions are only optional examples of the present disclosure, and are not used to limit the present disclosure. For those skilled in the art, various modifications and changes can be made to the present disclosure. Any modifications, equivalent substitutions, improvements and the like made within the spirit and principle of the present disclosure should be covered within the scope of protection of the present disclosure.

### INDUSTRIAL APPLICABILITY

The bifunctional macrocyclic chelate provided in the present disclosure has four linkers, which can be coupled with one or more target molecules, to realize more precise targeted diagnosis or treatment. Meanwhile, the bifunctional macrocyclic chelate has four linkers, which can be coupled with one or more target molecules, to achieve more precise targeted diagnosis or treatment. Meanwhile, the bifunctional macrocyclic chelate has relatively high selectivity and coordination capacity with divalent, trivalent, or tetravalent metal ions, and can form stable metal complexes with them. The metal complexes formed have good in vivo and in vitro stability, can specifically target tumors, and are mainly excreted through kidneys. The bifunctional macrocyclic chelate can complex most of the diagnostic or therapeutic metal ions, to achieve integration of radioactive diagnosis and treatment. Furthermore, diagnostic radiopharmaceuticals with a relatively long half-life period can also provide the possibilities of clinical efficacy monitoring and medication guidance for therapeutic medicines.

What is claimed is:

1. A bifunctional macrocyclic chelate, which is a compound of following Formula I or an isomer thereof, 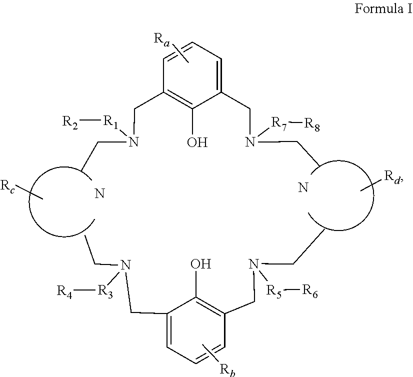
wherein R₁, R₃, R₅, and R₇ are each independently selected from substituted or unsubstituted alkyls or long-chain polymer groups,
R₂, R₄, R₆, and R₈ are each independently selected from functional groups capable of reacting with a target molecule and capable of coordinating with a metal ion,
Rₐ, R_{b}, R_{c}, and R_{d} are each independently selected from any one of H, substituted or unsubstituted alkyls, halogens, substituted or unsubstituted cyano groups, substituted or unsubstituted ester groups, substituted or unsubstituted alkoxys, substituted or unsubstituted amino groups, and substituted or unsubstituted aryls, and
ring

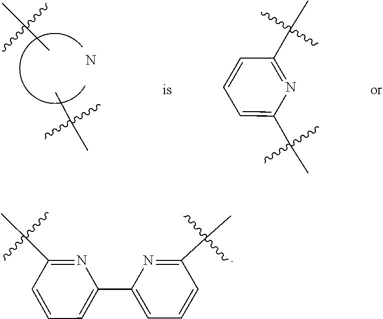

2. The bifunctional macrocyclic chelate according to claim 1, wherein R₁, R₃, R₅, and R₇ are each independently selected from straight-chain alkyls; and R₂, R₄, R₆, and R₈ are each independently selected from any one of amino groups, acid radical groups capable of forming organic acids, derivative groups of the amino groups, and derivative groups of the acid radical groups.

3. The bifunctional macrocyclic chelate according to claim 2, wherein R₁, R₃, R₅, and R₇ are each independently selected from C1-C20 unsubstituted straight-chain alkyls.

4. The bifunctional macrocyclic chelate according to claim 2, wherein R₁, R₃, R₅, and R₇ are each independently selected from any one of straight long-chain polymer groups containing amide groups and/or PEG groups.

5. The bifunctional macrocyclic chelate according to claim 1, wherein Rₐ, R_{b}, R_{c}, and R_{d} are each independently selected from H, halogens, C1-C20 substituted or unsubstituted alkyls, substituted or unsubstituted C1-C20 cyano groups, substituted or unsubstituted C1-C20 alkoxys, substituted or unsubstituted C2-C20 ester groups, substituted or unsubstituted C2-C20 amino groups, substituted or unsubstituted C3-C20 heteroaryls and C4-C20 non-heteroaryls.

6. The bifunctional macrocyclic chelate according to claim 1, wherein the bifunctional macrocyclic chelate is any one selected from compounds of following structural formulas:

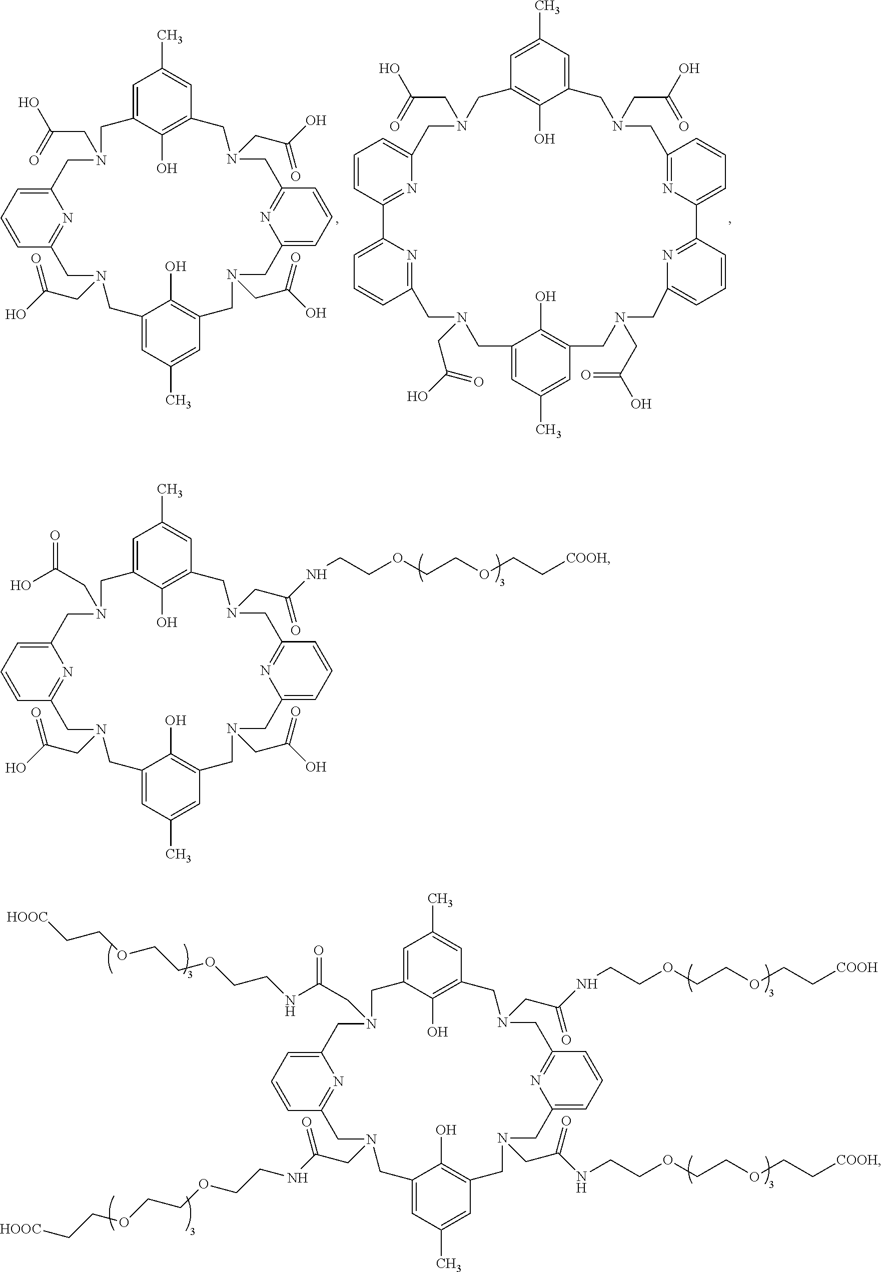

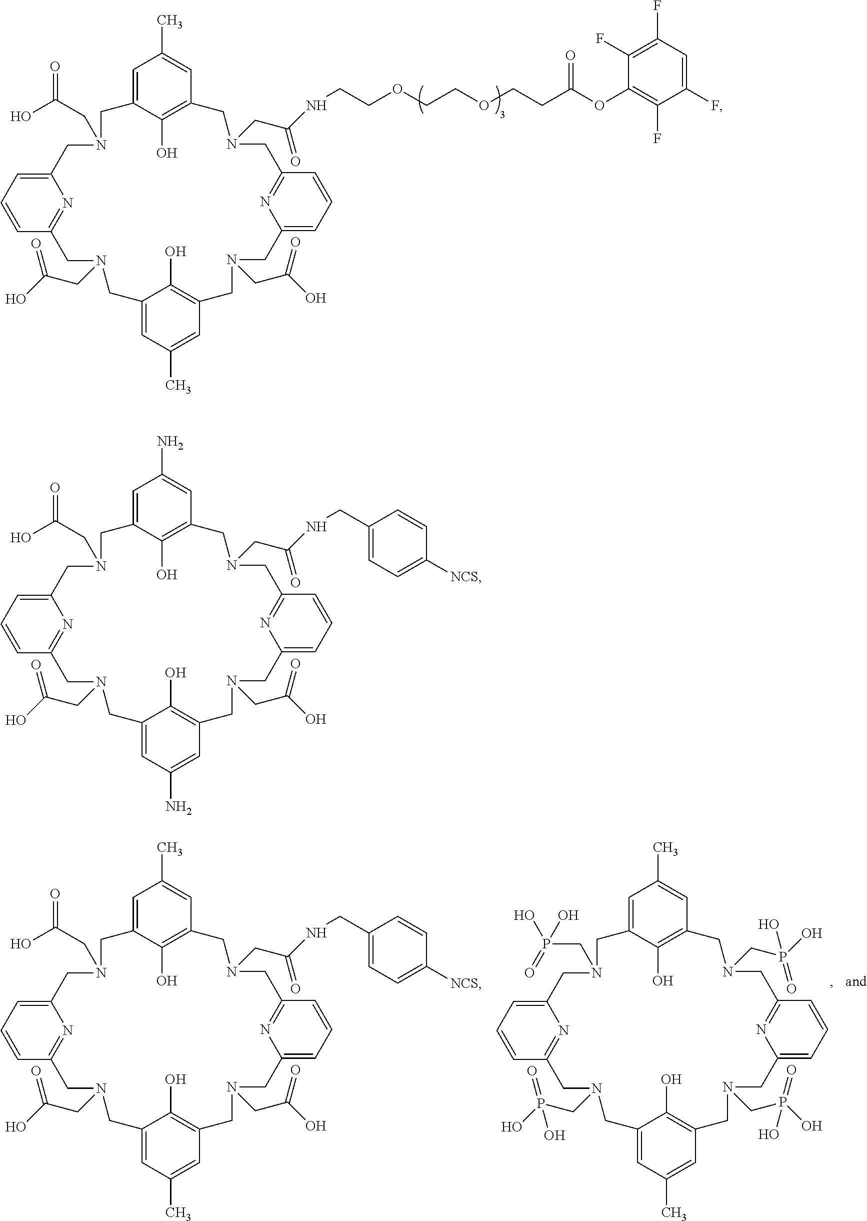

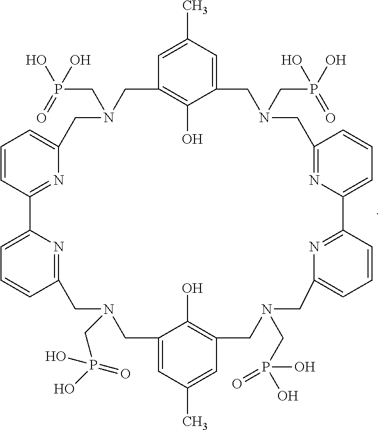 [SMILES: Cc1cc2c(O)c(c1)CN(CP(=O)(O)O)Cc1cccc(n1)-c1cccc(n1)CN(CP(=O)(O)O)Cc1cc(C)cc(c1O)CN(CP(=O)(O)O)Cc1cccc(n1)-c1cccc(n1)CN(CP(=O)(O)O)C2]

7. A metal complex, wherein the metal complex is a complex formed by chelation of a conjugate with a metal ion, and the conjugate is a compound formed by coupling of a target molecule with at least one group of R₂, R₄, R₆, and R₈ in the bifunctional macrocyclic chelate of claim 1.

8. The metal complex according to claim 7, wherein the target molecule is any one selected from antibodies, proteins, peptides, carbohydrates, nucleotides, oligonucleotides, oligosaccharides, vitamins, liposomes, small-molecule drugs or fragments or derivatives thereof.

9. The metal complex according to claim 7, wherein any one or two groups of R₂, R₄, R₆, and R₈ of the bifunctional macrocyclic chelate are coupled with the target molecule.

10. The metal complex according to claim 7, wherein the coupling process comprises: reacting the bifunctional macrocyclic chelate with a condensing agent and the target molecule under a condition of 0-100° C., wherein pH of a reaction system of the above reaction is 2-11, and the condensing agent is at least one of HOAt, HOBt, HATU, HBTU, DMAP, PyBOP, EDC, DCC, DIC, and NHS.

11. The metal complex according to claim 7, wherein the metal ion is a radioactive ion or a non-radioactive ion.

12. The metal complex according to claim 11, wherein the radioactive ion is selected from any one of Al[¹⁸F], ⁵¹Mn, ^{52m}Mn, ^{52g}Mn, ⁶⁴Cu, ⁶⁷Cu, ⁶⁷Ga, ⁶⁸Ga, ⁸⁹Zr, ⁸⁶Y, ⁹⁰Y, ^{99m}Tc, ¹¹¹In, ¹⁵³Sm, ¹⁶⁶Ho, ¹⁷⁷Lu, ¹⁸⁶Re, ¹⁸⁸Re, ²¹¹At, ²¹²Bi, ²¹²Pb, ²¹³Bi, ²²³Ra, ²²⁵Ac, and ²²⁷Th.

13. The metal complex according to claim 7, wherein the chelation process comprises: mixing a solution containing the conjugate with a solution containing the metal ion, adjusting pH of the solution to 3-11, and performing reaction under a condition of 0-110° C.

14. A method of treating tumors, comprising administering a therapeutically effective amount of the metal complex according to claim 7 to a subject in need thereof.

15. The method according to claim 14, wherein the target molecule is any one selected from antibodies, proteins, peptides, carbohydrates, nucleotides, oligonucleotides, oligosaccharides, vitamins, liposomes, small-molecule drugs or fragments or derivatives thereof.

16. The method according to claim 14, wherein any one or two groups of R₂, R₄, R₆, and R₈ of the bifunctional macrocyclic chelate are coupled with the target molecule.

17. The method according to claim 14, wherein the method further comprises a step of imaging, comprising administering a diagnostically effective amount of the metal complex according to claim 7 to a subject in need thereof, exposing the subject to a scanning device, and obtaining a scanning image of the subject.

18. The method according to claim 14, wherein the metal ion is a radioactive ion or a non-radioactive ion.

19. The method according to claim 14, wherein the radioactive ion is selected from any one of Al[¹⁸F], ⁵¹Mn, ^{52m}Mn, ^{52g}Mn, ⁶⁴Cu, ⁶⁷Cu, ⁶⁷Ga, ⁶⁸Ga, ⁸⁹Zr, ⁸⁶Y, ⁹⁰Y, ^{99m}Tc, ¹¹¹In, ¹⁵³Sm, ¹⁶⁶Ho, ¹⁷⁷Lu, ¹⁸⁶Re, ¹⁸⁸Re, ²¹¹At, ²¹²Bi, ²¹²Pb, ²¹³Bi, ²²³Ra, ²²⁵Ac, and ²²⁷Th.

20. The method according to claim 14, wherein the chelation process comprises: mixing a solution containing the conjugate with a solution containing the metal ion, adjusting pH of the solution to 3-11, and performing reaction under a condition of 0-110° C.
